(19) United States
(12) Patent Application Publication
(10) Pub. No.: US 2015/0004213 A1
(43) Pub. Date: Jan. 1, 2015

(54) Vaginal Drug Delivery Devices and Manufacturing Methods

(71) Applicant: VIMAC Ventures LLC, Boston, MA (US)

(72) Inventor: Eyal S. Ron, Lexington, MA (US)

(21) Appl. No.: 14/365,264

(22) PCT Filed: Dec. 16, 2012
(86) PCT No.: PCT/US2012/069973

§ 371 Date: Jun. 13, 2014

Related U.S. Application Data
(60) Provisional application No. 61/576,961, filed on Dec. 16, 2011.

(51) Int. Cl. A61K 38/09; B29C 45/17; A61K 9/00

(52) U.S. Cl. CPC A61K 38/09; A61K 9/0036; B29C 45/1775; B29K 2023/083
USPC 424/430; 514/10.4; 264/328.14

(57) ABSTRACT

Drug delivery devices (e.g., polymeric vaginal rings) and related methods of manufacture and treatment are disclosed herein. In some embodiments, a manufacturing process for drug delivery devices is disclosed that includes a compounding extrusion process and an injection molding process. The various manufacturing parameters associated with these processes can be optimized to produce a drug delivery device with a favorable release profile and other characteristics. In particular, reducing the energy introduced into the system during manufacture can unexpectedly result in drug delivery devices with improved release profiles, especially in the case of large molecule drugs or in devices with a relatively low drug loading or drug particle size.

### CROSS-REFERENCE TO RELATED APPLICATIONS

The present application claims priority to U.S. Provisional Application No. 61/576,961 filed on Dec. 16, 2011 and entitled “VAGINAL IMPLANTS AND METHODS,” which is hereby incorporated by reference in its entirety.

### FIELD

The present invention relates to drug delivery devices, methods of treatment, and methods for manufacturing drug delivery devices. In some embodiments, the present invention relates to polymeric vaginal rings and related methods of manufacture.

### BACKGROUND

Vaginal administration of drugs or other therapeutic agents can have several advantages over alternative delivery techniques. For example, the vagina includes highly perfused tissue with a well-developed blood supply, and vaginal delivery can be non-invasive and can avoid first-pass metabolism in the liver. In addition, administration of therapeutic agents via vaginal drug delivery can eliminate the need for therapies that involve painful injections or dosing regimens that are difficult or inconvenient to comply with.

A number of vaginal ring products have been developed which are configured for placement within the vagina and are configured to release one or more drugs. Exemplary vaginal rings are disclosed in U.S. Patent Application No. 2011/0280922, entitled “DEVICES AND METHODS FOR TREATING AND/OR PREVENTING DISEASES,” which is hereby incorporated by reference in its entirety. Such rings are generally formed from a mixture of one or more polymers, one or more drugs, and one or more excipients.

The processes used to manufacture vaginal rings and other drug delivery devices can impact the release profile and other parameters of the finished device. A need exists for improved drug delivery devices, methods of treatment, and methods of manufacturing drug delivery devices.

### SUMMARY

Drug delivery devices (e.g., polymeric vaginal rings) and related methods of manufacture and treatment are disclosed herein. In some embodiments, a manufacturing process for drug delivery devices is disclosed that includes a compounding extrusion process and an injection molding process. The various manufacturing parameters associated with these processes can be optimized to produce a drug delivery device with a favorable release profile and other characteristics. In particular, reducing the energy introduced into the system during manufacture can unexpectedly result in drug delivery devices with improved release profiles, especially in the case of large molecule drugs or in devices with a relatively low drug loading or drug particle size. Reducing the energy (e.g., by reducing extruder temperature, speed of extruder screw rotation, molding temperature, molding pressure, nozzle size, etc.) can result in less homogenous drug delivery devices. The drug particles are therefore less likely to be isolated from one another by the polymer and, as a result, more-effective formation of tortuous pathways occurs as the drug is released. Drug particles located in the inner areas of the device can then be released gradually through these pathways to obtain sustained release over extended periods.

In some embodiments, a method of manufacturing a drug delivery device is provided that includes mixing one or more drugs, one or more excipients, and one or more polymers to form a mixture, extruding the mixture using an extrusion system to form an extrudate, and injection molding at least a portion of the extrudate into a drug delivery device having a predetermined shape using an injection molding system. The mixture can be at least one of extruded through the extrusion system using an extrusion screw rotation speed between about 100 rpm and about 200 rpm, extruded through the extrusion system using a barrel temperature between about 70 degrees C. and about 90 degrees C., molded at a pressure between about 1400 bar and about 1700 bar, and injected into a mold through a nozzle having a cross-sectional area equal to that of a circle having a diameter between about 1.5 mm and about 2.5 mm.

The one or more drugs can be or can include leuprolide acetate, the one or more excipients can be or can include Polysorbate 80, and the one or more polymers can be or can include ethylene-vinyl-acetate (EVA) copolymer and polyethylene glycol (PEG). The one or more polymers can be or can include EVA 28-40, EVA 18-150, and PEG 4000. The mixture can be or can include EVA 28-40 at a weight percentage of 44.3, PEG 4000 at a weight percentage of 8.0, Polysorbate 80 at a weight percentage of 1.0, EVA 18-150 at a weight percentage of 44.3, and leuprolide acetate at a weight percentage of 2.4. The mixture can be fed into the extrusion system at a rate of about 0.5 kg/hr to about 2.0 kg/hr, at a rate of less than about 2.0 kg/hr, or at a rate of about 1.0 kg/hr.

The mixture can be extruded through the extrusion system using a barrel configuration that includes a first open section where the mixture is fed, a second closed section, a third closed section, a fourth open section where venting occurs, and a fifth closed section. The mixture can be extruded through the extrusion system using an element configuration of GFF 2-30-90 at the feed, followed by GFA 2-30-60, followed by GFA 2-20-30, followed by KB4 2-15-60 RE, followed by GFA 2-30-60, followed by KB4 2-15-30 RE, followed by GFA 2-30-60, followed by GFA 2-30-30, followed by GFA 2-15-60, followed by GFA 2-15-30.

The mixture can be extruded through the extrusion system using an extrusion screw rotation speed between about 100 rpm and about 200 rpm, using an extrusion screw rotation speed less than about 200 rpm, or using an extrusion screw rotation speed of about 150 rpm. The mixture can be extruded through the extrusion system using a barrel temperature between about 70 degrees C. and about 90 degrees C., using a barrel temperature less than about 90 degrees C., or using a barrel temperature of about 80 degrees C. The mixture can be extruded through a nozzle having a cross-sectional area equal to that of a circle having a diameter between about 2.0 mm and about 4.0 mm, having a diameter of at least about 2.5 mm, or having a diameter of about 3.0 mm. The mixture can be fed into the extrusion system at a rate of about 1.0 kg/hr, extruded through the extrusion system using an extrusion screw rotation speed of about 150 rpm, extruded through the extrusion system using a barrel temperature of about 80 degrees C., and extruded through a nozzle having a cross-sectional area equal to that of a circle having a diameter of about 3.0 mm.

The method can also include pelletizing the extrudate before said injection molding. The method can also include blending the extrudate after said pelletizing and before said injection molding. The extrudate can be advanced into a mold of the injection molding system at a rate between about 50 mm per second and about 150 mm per second, at a rate less than about 125 mm per second, or at a rate of about 100 mm per second. The extrudate can be molded at a pressure between about 1400 bar and about 1700 bar, at a pressure less than about 1600 bar, or at a pressure of about 1550 bar. The extrudate can be molded using a barrel temperature between about 75 degrees C. and about 95 degrees C., using a barrel temperature less than about 90 degrees C., or using a barrel temperature of about 80 degrees C. The extrudate can be molded using a mold temperature between about 45 degrees C. and about 65 degrees C., using a mold temperature less than about 60 degrees C., or using a mold temperature of about 55 degrees C.

The extrudate can be injected into a mold through a nozzle having a cross-sectional area equal to that of a circle having a diameter between about 1.5 mm and about 2.5 mm, having a diameter of at least about 1.75 mm, or having a diameter of about 2.0 mm. The extrudate can be advanced into a mold of the injection molding system at a rate of about 100 mm per second, molded at a pressure of about 1550 bar, molded using a barrel temperature of about 80 degrees C., molded using a mold temperature of about 55 degrees C., and injected into a mold through a nozzle having a cross-sectional area equal to that of a circle having a diameter of about 2.0 mm. The predetermined shape can be or can include a ring. The ring can have a minor diameter of about 4 mm and a major diameter of about 54 mm.

In some embodiments, a drug delivery device is provided that includes a ring-shaped body that includes leuprolide acetate, Polysorbate 80, ethylene-vinyl-acetate (EVA) copolymer, and polyethylene glycol (PEG). When placed in a vaginal tract of a patient, the ring-shaped body can be configured to release the leuprolide acetate at a rate of at least about 0.1 mg/day for a period of at least about 10 days. When placed in the vaginal tract of the patient, the ring-shaped body can be configured to release the leuprolide acetate at a rate of at least about 0.15 mg/day. When placed in the vaginal tract of the patient, the ring-shaped body can be configured to release the leuprolide acetate for a period of at least about 28 days.

In some embodiments, a drug delivery device is provided that includes a ring-shaped body that includes a mixture of one or more drugs, one or more excipients, and one or more polymers. The body is formed using an extrusion process followed by an injection molding process, the extrusion process or the injection molding process including at least one of extruding the mixture using an extrusion screw rotation speed between about 100 rpm and about 200 rpm, extruding the mixture using a barrel temperature between about 70 degrees C. and about 90 degrees C., molding the mixture at a pressure between about 1400 bar and about 1700 bar, and injecting the mixture into a mold through a nozzle having a cross-sectional area equal to that of a circle having a diameter between about 1.5 mm and about 2.5 mm.

The one or more drugs can be or can include leuprolide acetate, the one or more excipients can be or can include Polysorbate 80, and the one or more polymers can be or can include ethylene-vinyl-acetate (EVA) copolymer and polyethylene glycol (PEG). The one or more polymers can be or can include EVA 28-40, EVA 18-150, and PEG 4000. The body can be or can include EVA 28-40 at a weight percentage of 44.3, PEG 4000 at a weight percentage of 8.0, Polysorbate 80 at a weight percentage of 1.0, EVA 18-150 at a weight percentage of 44.3, and leuprolide acetate at a weight percentage of 2.4.

The extrusion process can include feeding the mixture into an extrusion system at a rate of about 0.5 kg/hr to about 2.0 kg/hr. The extrusion process can include extruding the mixture using a barrel configuration that includes a first open section where the mixture is fed, a second closed section, a third closed section, a fourth open section where venting occurs, and a fifth closed section. The extrusion process can include extruding the mixture using an element configuration of GFF 2-30-90 at the feed, followed by GFA 2-30-60, followed by GFA 2-20-30, followed by KB4 2-15-60 RE, followed by GFA 2-30-60, followed by KB4 2-15-30 RE, followed by GFA 2-30-60, followed by GFA 2-30-30, followed by GFA 2-15-60, followed by GFA 2-15-30. The extrusion process can include extruding the mixture using an extrusion screw rotation speed between about 100 rpm and about 200 rpm. The extrusion process can include extruding the mixture using a barrel temperature between about 70 degrees C. and about 90 degrees C. The extrusion process can include extruding the mixture through a nozzle having a cross-sectional area equal to that of a circle having a diameter between about 2.0 mm and about 4.0 mm.

The injection molding process can include advancing the mixture into a mold at a rate between about 50 mm per second and about 150 mm per second. The injection molding process can include molding the mixture at a pressure between about 1400 bar and about 1700 bar. The injection molding process can include molding the mixture using a barrel temperature between about 75 degrees C. and about 95 degrees C. The injection molding process can include molding the mixture using a mold temperature between about 45 degrees C. and about 65 degrees C. The injection molding process can include injecting the mixture into a mold through a nozzle having a cross-sectional area equal to that of a circle having a diameter between about 1.5 mm and about 2.5 mm. The body can have a minor diameter of about 4 mm and a major diameter of about 54 mm.

The present invention further provides devices, systems, and methods as claimed.

### BRIEF DESCRIPTION OF THE DRAWINGS

The invention will be more fully understood from the following detailed description taken in conjunction with the accompanying drawings, in which:

FIG. 1 is a plan view of a ring-shaped drug delivery device;

FIG. 2 is a plan view of a ring-shaped drug delivery device having a plurality of segments;

FIG. 3 is a schematic diagram of a system for manufacturing drug delivery devices;

FIG. 4A is a schematic diagram of a barrel and element configuration for the extrusion system shown in FIG. 3;

FIG. 4B is a schematic diagram of another barrel and element configuration for the extrusion system shown in FIG. 3;

FIG. 4C is a schematic diagram of another barrel and element configuration for the extrusion system shown in FIG. 3;

FIG. 5 is a plan view of first and second mold plates for the injection molding system shown in FIG. 3;

FIG. 6 is a graph of release kinetics for drug delivery devices having different drug particle sizes;

FIG. 7 is a graph of release kinetics for drug delivery devices having different drug loadings;

FIG. 8 is a graph of release kinetics for drug delivery devices having different drug molecular weights;

FIG. 9 is a graph of release kinetics for drug delivery devices having different formulations;

FIG. 10 is a graph of release kinetics for drug delivery devices having different formulations;

FIG. 11 is a graph of drug potency as a function of extrusion time in various portions of a system for manufacturing drug delivery devices;

FIG. 12 is a graph of release kinetics for drug delivery devices manufacturing using different extrusion screw speeds;

FIG. 13 is a graph of release kinetics for drug delivery devices manufacturing using different extrusion screw speeds;

FIG. 14 is a graph of release kinetics for drug delivery devices manufacturing using different techniques and/or different mold temperatures;

FIG. 15 is a graph of device stiffness for drug delivery devices manufacturing using different techniques;

FIG. 16A is a common transmission image of a raw extrudate;

FIG. 16B is a common transmission image of a drug delivery device manufactured using high pressure injection molding;

FIG. 16C is a common transmission image of a drug delivery device manufactured using low pressure injection molding;

FIG. 16D is a polarized light image of a raw extrudate;

FIG. 16E is a polarized light image of a drug delivery device manufactured using high pressure injection molding;

FIG. 16F is a polarized light image of a drug delivery device manufactured using low pressure injection molding; and

FIG. 17 is a schematic diagram of an exemplary method of producing pelletized and blended extrudate pellets for manufacturing drug delivery devices.

### DETAILED DESCRIPTION

Certain exemplary embodiments will now be described to provide an overall understanding of the principles of the structure, function, manufacture, and use of the methods, systems, and devices disclosed herein. One or more examples of these embodiments are illustrated in the accompanying drawings. Those skilled in the art will understand that the methods, systems, and devices specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Drug delivery devices (e.g., polymeric vaginal rings) and related methods of manufacture and treatment are disclosed herein. In some embodiments, a manufacturing process for drug delivery devices is disclosed that includes a compounding extrusion process and an injection molding process. The various manufacturing parameters associated with these processes can be optimized to produce a drug delivery device with a favorable release profile and other characteristics. In particular, reducing the energy introduced into the system during manufacture can unexpectedly result in drug delivery devices with improved release profiles, especially in the case of large molecule drugs or in devices with a relatively low drug loading or drug particle size. Reducing the energy (e.g., by reducing extruder temperature, speed of extruder screw rotation, molding temperature, molding pressure, nozzle size, etc.) can result in less homogenous drug delivery devices. The drug particles are therefore less likely to be isolated from one another by the polymer and, as a result, more-effective formation of tortuous pathways occurs as the drug is released. Drug particles located in the inner areas of the device can then be released gradually through these pathways to obtain sustained release over extended periods.

It should be appreciated that the notion of reducing the energy inputs into an extrusion and injection molding process is in direct contradiction with the conventional wisdom, which was to use higher temperatures, higher pressures, and vigorous mixing in an effort for more-consistent and more-uniform finished products. This was perhaps due to the fact that early polymeric drug delivery devices focused on delivering small molecule drugs, which were capable of being released by diffusion through the polymer, as opposed to large molecule drugs which generally require tortuous pathway formation in order to obtain release over an extended duration. Reducing the energy inputs, as disclosed herein, unexpectedly resulted in drug delivery devices with improved release kinetics and the ability to release large and small molecule drugs over an extended period of time.

The terms “therapeutic agents,” “active agents,” “drugs,” and the like are generally used interchangeably herein to refer to any functional agent that can be delivered to a human or animal patient, including hormones, stem cells, gene therapies, chemicals, compounds, small and large molecules, dyes, antibodies, viruses, physiologically or pharmacologically active agents that produce a local and/or systemic effect, and so forth.

Drug Delivery Devices

An exemplary drug delivery device can include at least one polymeric form, e.g., a ring, a rod, a string, or a thread, that includes at least one, two, three, or more segments, with a least one segment including a thermoplastic polymer and a drug. The device can release the drug over time when placed in a patient, e.g., in a vagina of a patient. A segment can have a substantially uniform composition (e.g., of both the drug and polymer) throughout. In other words, the segment can, in some embodiments, lack any rate-controlling membrane or concentrated drug reservoir. The drug delivery device can be capable of releasing the drug over time when placed in the vaginal cavity of a patient. For example, the device can be capable of delivering a pharmaceutically-effective amount of one or more contraceptive agents intravaginally for about 1 day or more, about 1 week, about 1 month, about 3 months, or about 6 months or more, with or without replacing the device once placed within the vagina.

The drug delivery device can include one or more segments which each can include the same active agent or each can include a different active agent. Each segment can optionally include further active agents, or, in the case of a device that includes two or more segments, different segments can each include a different drug, or one or more segments can include a drug and/or another therapeutic agent or an agent that augments delivery of an active agent, or one or more segments can include an active agent with another segment including another agent, or two or more segments can include the same active agent (e.g., in the same weight percentage or a different weight percentage), or one, two, or more segments can include no active agent. Similarly, a device that includes two or more segments can include a first segment with a different thermoplastic polymer than a second segment. For example, a first segment can include a thermoplastic polymer with a different release rate than a second segment (which can result from, e.g., a different polymer or a different percentage of monomer, e.g., a different percentage of vinyl acetate in ethylene vinyl acetate co-polymer.)

In some embodiments, a unitary segment can be formed in a ring shape. In other embodiments, two, three, or more segments can be joined end to end to form a ring shape. For example, at least one end of a segment can be attached to the end of another unitary segment by a coupling means, such as an adhesive material or by annealing the ends of the segments to same or different thermoplastic polymers.

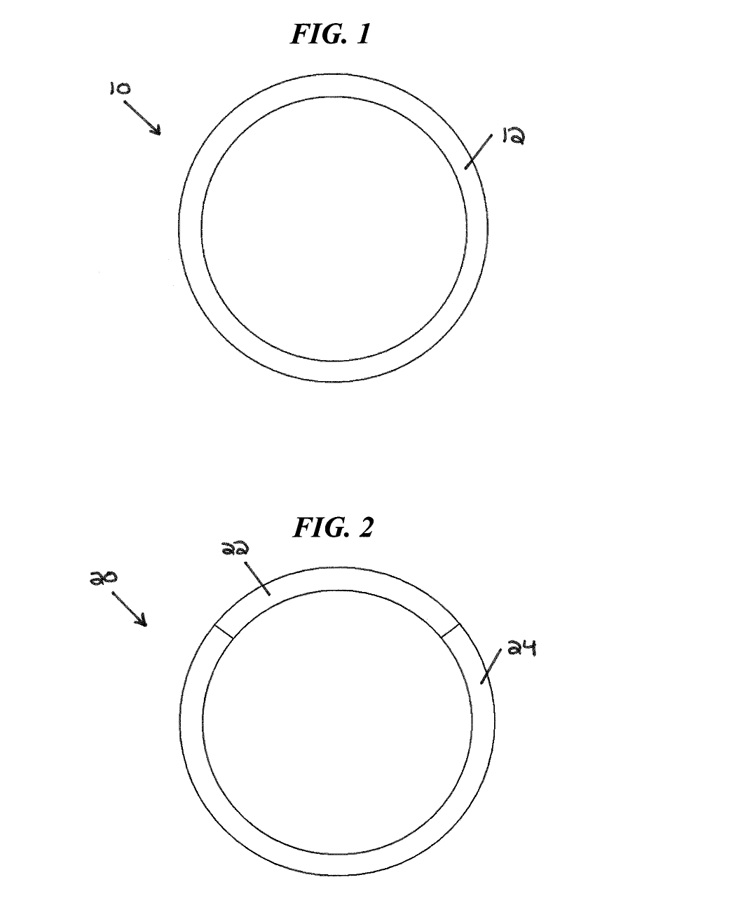

FIG. 1 illustrates an exemplary embodiment of a drug delivery device 10 that includes a body 12 sized, shaped, configured, and adapted for placement in the vaginal tract of a human. The body 12 can be formed of a polymer that releases one or more drugs by diffusion or other transport mechanism into the vaginal tract of the patient. FIG. 2 illustrates an exemplary embodiment of a drug delivery device 20 that includes two unitary cylindrical segments 22 and 24 which are connected to each other by a coupling means (not shown) to form a continuous ring. The two segments 22, 24 can also be directly fused without the need for a coupling means, or alternatively, the ring can be formed from one segment, e.g., as shown in FIG. 1, which may or may not eliminate the need for a coupling means. Although the illustrated devices include one or two segments, drug delivery devices can include three, four, five, six, or more segments. The number and size of the segments used for a particular application will depend, for example, on the number of drugs to be delivered, the dosages of the drugs, and the need for one or more placebo segments to prevent diffusion and interaction of the drugs within the device. For example, an exemplary ring can consist essentially of a unitary segment that includes or, in some embodiments, consists essentially of, ethylene vinyl acetate (EVA) copolymer and/or polyethylene glycol (PEG), and an effective amount of therapeutic peptide and optionally, a pharmaceutically-acceptable excipient.

In some embodiments, delivery devices that include EVA with or without PEG and a therapeutic peptide, and optionally an excipient such as a surfactant and/or an emulsifier such as a nonionic surfactant, e.g., Tween (for example Tween 80 or polysorbate 80) can be a standalone implantable body having a uniform cross-section at all points along a length of the implantable body. In some embodiments, the drug delivery device can have a cross-sectional diameter substantially identical to a cross-sectional diameter of the implantable body, e.g., at all points along a length of device (e.g., an entire ring). Thus, the device can lack any kind of rate-controlling membrane or concentrated drug reservoir.

The drug delivery devices disclosed herein can also be formed as part of an absorbent tampon, or in the shape of a wafer or suppository. It will be appreciated that the drug delivery devices disclosed herein can be manufactured in a variety of shapes, sizes, and dimensions, depending upon the particular mammal to be treated, as well as the nature and severity of the condition to be treated.

In some embodiments, the drug delivery device can include two or more unitary segments, wherein a first segment includes a uniform mixture of a drug-permeable polymeric substance (e.g., EVA or a combination of EVA and PEG, and optionally an excipient such as Tween 80) and a first active agent, and a second segment includes a second drug-permeable polymeric substance and a second active agent, with an optional third segment which can include another active agent which can be the same or different than that in the second segment. At least two of the segments can include a different active agent. In some embodiments, the first and second permeable polymeric substance can be the same, e.g., a thermoplastic polymer, such as an EVA copolymer. When the drug delivery device includes one or more polymeric shapes for the release of two active agents (e.g., both an antiandrogen and a contraceptive agent, or isotretinoin and a contraceptive agent) the system can release each agent in a substantially constant ratio over a prolonged period of time.

In some embodiments, the drug delivery device can be a vaginal ring that includes EVA co-polymer and an amount of an active agent appropriate for systemic delivery over time to patient when placed in the vagina. The vaginal ring can be formed from one unitary segment, and for example, can consist essentially of EVA co-polymer and an active agent and optionally one or more pharmaceutically acceptable excipients. Such a ring can be capable of delivering an active agent to a patient with a reduced and/or delayed peak serum concentration, for example, as compared to a patient administered the active agent as a depot injection (for example Lupron Depot® having 3.75 mg, 7.5 mg, 11.25 mg, 22.5 mg, and/or 30 mg of leuprolide acetate in a polylactic acid depot, or a depot composition that comprises about 11.25 mg or 22.5 mg of leuprolide acetate and polylactic acid).

The drug delivery device can capable of delivering an active agent to a patient with a decrease in peak serum concentration, for example, as compared to a patient administered the active agent as a depot injection, but can deliver appropriate amounts of the active agent effective to achieve a treatment over 3 days, 1 week, 1 month, or more. Such an exemplary vaginal ring, that effectively includes an active agent and EVA co-polymer (and optionally a pharmaceutically-acceptable excipient) can deliver systemically, in some embodiments, a non-linear increase in the amount of active agent to a patient, with respect to the amount of active agent present in the ring, which, in some embodiments, may not be achievable if other active agents and/or polymers and/or peptides are present in the device.

For example, a drug delivery device can include unitary segments that include EVA co-polymer and an active agent wherein an increase in dose of active agent present in the ring results in a greater increase of peak serum levels of the active agent in a patient than the expected serum level resulting from the increased dosage in the device. For example, a doubling of the dose of active agent in the device can lead to an about three-fold increase in peak serum levels of the active agent in patient (once placed in the vagina of the patient).

Systemic administration using a vaginal device, e.g., a ring, can result in a peak serum concentration of the active agent (e.g., leuprolide) in a patient at about 12 to about 22 hours, e.g., about 14 to about 17 hours, about 15 or about 16 hours after insertion of the device. The device can include about 18 mg to about 100 mg of therapeutic leuprolide, e.g., about 18 mg, about 36 mg, or about 54 mg or more. For example, upon administration and once inserted in a patient, a device can result in a serum level of leuprolide in the patient of about 0.01 ng/mL to about 2.0 ng/mL, or about 0.1 ng/mL to about 1.0 ng/mL, e.g., about 0.6 ng/mL or about 1.0 ng/mL, after about 12 hours, after about 18 hours, after about 20 hours, or even after about 1 day. The device can produce an exemplary peak leuprolide level in a patient of about 0.5 ng/mL to about 4 ng/mL, at for example, about 16 hours after patient insertion.

In order to achieve constant levels of each of one or more active agents and avoid the inefficiencies of concentration peaks and valleys, active agents can be released from a delivery device at a rate that does not substantially change with time (so called zero-order release). Preferably, the initial dose of an active agent is the therapeutic dose, which is maintained by the delivery device.

In some embodiments, the drug delivery device can provide for substantially “zero order kinetic” active agent administration, in which an active agent is released in a steady state, thus providing a corresponding predictable absorption and metabolism of the active agent in the body tissues. For example, contemplated therapeutic devices that include leuprolide, upon insertion into a patient's (e.g., a human) vagina, can result in a peak serum concentration of leuprolide about 12 to about 22 hours, e.g., about 15, 16, or 17 hours after insertion. Such a peak serum concentration in a patient can be less than that of a patient administered a leuprolide depot concentration by injection (such as a depot composition having 22.5 mg or 11.5 mg of leuprolide, e.g., Lupron® depot). For example, after insertion of a device that includes leuprolide, a patient can have peak serum levels of FSH and/or LH about 12 to about 18 hours after insertion, e.g., at about 15 or about 16 hours. Such peak levels of FSH and LH can occur later in a patient as compared to the time peak levels of FSH and LH occur in a patient administered a depot composition of leuprolide (such as Lupron® depot). In some embodiments, a contemplated therapeutic device can release about 5 μg to about 150 μg/day, e.g., about 10 μg/day of a therapeutic protein, e.g., leuprolide, upon insertion into the vagina of a patient.

Using the delivery devices disclosed herein, delivery of active agents can be “targeted” to the specific body organ where the intended therapeutic effect is desired, bypassing other organs such as liver, in which unintended effects can occur. Thus, the efficient metabolic and therapeutic use of one or more active agents can be enhanced, and the development of adverse metabolic side effects can be reduced.

Drugs

The devices disclosed herein can deliver any of a variety of active agents and combinations of active agents, e.g., any of those disclosed in U.S. Patent Application No. 2011/0280922, entitled “DEVICES AND METHODS FOR TREATING AND/OR PREVENTING DISEASES,” which is hereby incorporated by reference in its entirety. Exemplary active agents include isotretinoin, antiandrogens, therapeutic peptides, leuprolide, contraceptive agents, antibacterial agents, cholesterol lowering medications, beta-blockers, nitroglycerin, calcium channel blockers, aspirin, COPD and/or asthma treatment agents, chronic kidney disease treatment agents, anti-migraine drugs, anti-nausea drugs, analgesics, estrogenic steroids, progestation steroids, interferon, anti-angiogenesis factors, antibodies, antigens, polysaccharides, growth factors, and hormones.

A drug to be delivered can have a molecular weight of between about 50 and about 20000, and preferably between about 50 and about 2000, and more preferably between about 200 and about 1300. The dosage unit amount for conventional beneficial drugs as described herein is well known in the art, as disclosed for example in Remington's Pharmaceutical Science (Fourteenth ed., Part IV, Mack Publishing Co., Easton, Pa., 1970), which is hereby incorporated by reference in its entirety. The amount of drug incorporated in the drug delivery device can vary depending on the particular drug, the desired therapeutic effect, and the time span for which the device provides therapy. Since the drug delivery devices disclosed herein can provide dosage regimes for therapy for a variety of applications and indications, there is no critical upper limit on the amount of drug incorporated in the device. Similarly, the lower limit will depend on the activity of the drug and the time span of its release from the device.

The relative amount(s) of the agents(s) to be released can be modified over a wide range depending upon the active agent to be administered or the desired effect. Generally, the agent can be present in an amount which will be released over controlled periods of time, according to predetermined desired rates, which rates are dependent, at least in part, upon the initial concentration of the active substance in the polymer. In one embodiment, a rate can also depend upon the level of ultrasonic energy to which it is subjected. This necessarily implies a quantity of active substance greater than the standard single dosage. Suitable proportions can range from about 0.01 to 50 parts by weight of the active substance to between about 99.99 and about 50 parts by weight of the polymer, preferably between about 10 and about 30 parts by weight in the case of an active agent to be implanted to give 100 parts per weight of the final system. The polymer in the composition to be released can be admixed in any convenient manner, for example by mixing the components as powders and subsequently forming the mixture into a desired shape such as by thermal forming at a temperature less than that which the composition will become degraded and at which the polymer has desired morphological properties.

Excipients

As noted above, the drug or drugs can be present in the device in combination with a biocompatible excipient or carrier acceptable for application of the drug to the vaginal epithelium. Exemplary excipients include wetting agents, surfactants, polaxomers, carbomers, polyvinyl alcohol, silicon dioxide, sodium carboxymethyl cellulose, emulsifiers, nonionic surfactants, Tween, Tween 80, polysorbate 80, and/or combinations thereof. Other suitable excipients are described in the Compendium of Pharmaceutical Excipients for Vaginal Formulations, Sanjay Garg et al., Pharmaceutical Technology, Drug Delivery 2001, the entire contents of which are incorporated herein by reference.

Other pharmaceutically acceptable excipients include a-lipoic acid, α-tocopherol, ascorbyl palmitate, benzyl alcohol, biotin, bisulfites, boron, butylated hydroxyanisole, butylated hydroxytoluene, ascorbic acid, carotenoids, calcium citrate, acetyl-L-carnitine, chelating agents, chondroitin, chromium, citric acid, coenzyme Q-10, cysteine, cysteine hydrochloride, 3-dehydroshikimic acid, EDTA, ferrous sulfate, folic acid, fumaric acid, alkyl gallates, garlic, glucosamine, grape seed extract, gugul, magnesium, malic acid, metabisulfite, N-acetyl cysteine, niacin, nicotinomide, nettle root, ornithine, propyl gallate, pycnogenol, saw palmetto, selenium, sodium bisulfite, sodium metabisulfite, sodium sulfite, potassium sulfite, tartaric acid, thiosulfates, thioglycerol, thiosorbitol, tocopherol, tocopherol acetate, tocopherol succinate, tocotrienal, d-.alpha.-tocopherol acetate, vitamin A, vitamin B, vitamin C, vitamin D, vitamin E, zinc, carbohydrates, and combinations thereof.

For example, excipients can include one or more of sodium acetate, sodium carbonate, citrate, glycylglycine, histidine, glycine, arginin, sodium dihydrogen phosphate, disodium hydrogen phosphate, sodium phosphate, and tris (hydroxymethyl)-aminomethane, bicine, tricine, magic acid, succinate, maleic acid, fumaric, acid, tartaric acid, citric acid, aspartic acid, ethylenediaminetetraacetic acid (EDTA), and combinations thereof. In some embodiments, the device can include one or more carbohydrates and/or citric acid and/or one or more cellulose ethers (such as hydroxypropyl methylcellulose). The drugs included in the device can be absorbable through the vaginal mucosa and thereby transmitted via venous and lymphatic channels to the uterus or to the general blood circulation.

Polymers

Exemplary polymers for use in the drug delivery devices disclosed herein can include olefin and vinyl-type polymers, carbohydrate-type polymers, condensation-type polymers, rubber-type polymers, and/or organosilicon polymers. Other exemplary polymers that can be used include poly(ethylene-vinyl acetate), poly(methylacrylate), poly(butylmethacrylate), plasticized poly(vinylchloride), plasticized nylon, plasticized soft nylon, plasticized poly(ethylene terephthalate), poly(ethylene), poly(acrylonitrile), poly(trifluorochloroethylene), poly(4,4′-isopropylene-diphenylene carbonate), poly(ethylenevinyl esters), poly(vinyl chloridediethyl fumarate), poly(esters of acrylic and methacrylic), cellulose acetate, cellulose acylates, partially hydrolyzed poly(vinyl acetate), poly(vinyl butyral), poly(amides), poly(vinyl carbonate), poly(urethane), poly(olefins), and the like and combinations thereof. These polymers and their physical properties are known in the art and can be synthesized according to the procedures disclosed, for example, in Encyclopedia of Polymer Science and Technology (Interscience Publishers, Inc., New York, 1971) Vol. 15, pp. 508-530; Polymers (1976), Vol. 17, 938-956; Technical Bulletin SCR-159, 1965, Shell Corp., New York; and references cited therein; and in Handbook of Common Polymers, Scott and Roff (CRC Press, Cleveland, Ohio, 1971), which are each hereby incorporated by reference in their entirety.

In some embodiments, the polymer can be capable of being degraded by ultrasonic energy such that any incorporated agent is released at a rate within a desired release range, or, in the case of nondegradable polymers, release is enhanced. Representative suitable polymers for such embodiments can include polyesters such as poly(lactic acid), poly(lactic-co-glycolic acid), and/or polyanhydrides having the formula described in U.S. Pat. No. 4,657,543 (Langer et al.), which is hereby incorporated by reference in its entirety. The monomers in any copolymer can be distributed regularly or at random. For example, an anhydride linkage can be highly reactive toward hydrolysis, and therefore, in some embodiments, it can be preferable that the polymer backbone be hydrophobic in order to attain the heterogeneous erosion of the encapsulated composition.

Hydrophobicity of polymers can be regulated easily, for example, by regulating the concentration of aromatic moieties in the linking backbone, or by monitoring the monomer ratio in the copolymer. In some embodiments, the polymeric backbone can include or can be formed from an acid such as 1-phenylamine, tryptophan, tyrosine or glycine. Other polymers include ethylene-vinyl acetate, poly(lactic acid), poly(glutamic acid), polycaprolactone, lactic/glycolic acid copolymers, polyorthoesters, polyamides or the like. Non-degradable polymers include ethylene-vinyl acetate, silicone, hydrogels such as polyhydroxyethylmethacrylate, polyvinyl alcohol, and the like.

In addition to providing appropriate release properties, a drug permeable polymeric substance can be formed from a compatible, non-absorbable, non-toxic polymeric substance that does not significantly induce a significant tissue reaction at the site of placement in the vaginal tract of the female mammal.

In some embodiments, one more segments comprise ethylene-vinyl acetate (EVA) copolymer and/or polyethylene glycol (PEG). Suitable EVA polymers include, for example, the EVA material manufactured by Aldrich Chemical Co. (Cat. No. 34, 050-2); Evatane® with the designations 28-150, 28-399, and 28-400, supplied by ICI and 28.420, and in particular 28.25 and 33.25 supplied by Atochem; and Elvax® with the designations 310, 250, 230, 220, and 210, supplied by Du Pont de Nemours. Exemplary EVA polymers can include a mixture of EVA having a 27-29 weight percent vinyl acetate content and EVA having a 17-19 weight percent vinyl acetate content, e.g., Evatane® 18-150 and 28-25.

One or more segments can, in some embodiments, also include PEG, such as a PEG with a weight average molecular weight of about 2000 Da to about 8000 Da, e.g., about 3600 Da to about 4400 Da (e.g., 4000 Da).

In drug delivery devices that include EVA, the drug release can be determined, at least in part, by the vinyl acetate content of polymeric substance. In some embodiments, the EVA copolymers used in the device can have a vinyl acetate content of about 4 to 80% by weight of the total, and a melt index of about 0.1 to 1000 grams per ten minutes. Melt index is the number of grams of polymer which can be forced through a standard cylindrical orifice under a standard pressure at a standard temperature, and thus is inversely related to the molecular weight of the polymer. In some embodiments, the EVA has a vinyl acetate content of about 4 to 50% by weight and a melt index of about 0.5 to 250 grams per ten minutes. For example, a unitary segment can include about 40% weight percent vinyl acetate and/a melt index of about 48 to about 62 grams per ten minutes, e.g., 57 grams per ten minutes, at e.g., 190 degrees C./2.16 kg. In some embodiments, the device can include Evatane® 40-55, described at www.arkema-inc.com/tds/1126.pdf, hereby incorporated by reference in its entirety. In some embodiments, the amount of vinyl acetate present in a finally-processed ring is minimal or substantially undetectable.

In general, the rate of passage of an active agent through the polymer can be dependent on the molecular weight and solubility of the agent therein, as well as on the vinyl acetate content of the polymer, and in some embodiments, selection of particular EVA compositions can depend on the particular active agent to be delivered. For example, by varying the composition and properties of the EVA, the dosage rate per area of the device can be controlled, for example, different segments of a polymeric shape can each include different compositions of EVA. Thus, devices of the same surface area can provide different dosage of an active agent by varying the characteristics of the EVA copolymer. The release of the active agent by a drug delivery device comprising EVA can also be controlled by the surface area of the segment. For example, the length and/or circumference of the segment can be increased, in some embodiments, to increase the rate of release of the active agent.

Methods of Manufacture

The drug delivery devices disclosed herein can be manufactured using a variety of processes and techniques. The various manufacturing parameters associated with these processes can be optimized to produce a drug delivery device with a favorable release profile and other characteristics. In particular, reducing the energy introduced into the system during manufacture can unexpectedly result in drug delivery devices with improved release profiles, especially in the case of large molecule drugs or in devices with a relatively low drug loading or drug particle size. Reducing the energy (e.g., by reducing extruder temperature, speed of extruder screw rotation, molding temperature, molding pressure, nozzle size, etc.) can result in less homogenous drug delivery devices. The drug particles are therefore less likely to be isolated from one another by the polymer and, as a result, more-effective formation of tortuous pathways occurs as the drug is released. Drug particles located in the inner areas of the device can then be released gradually through these pathways to obtain sustained release over extended periods.

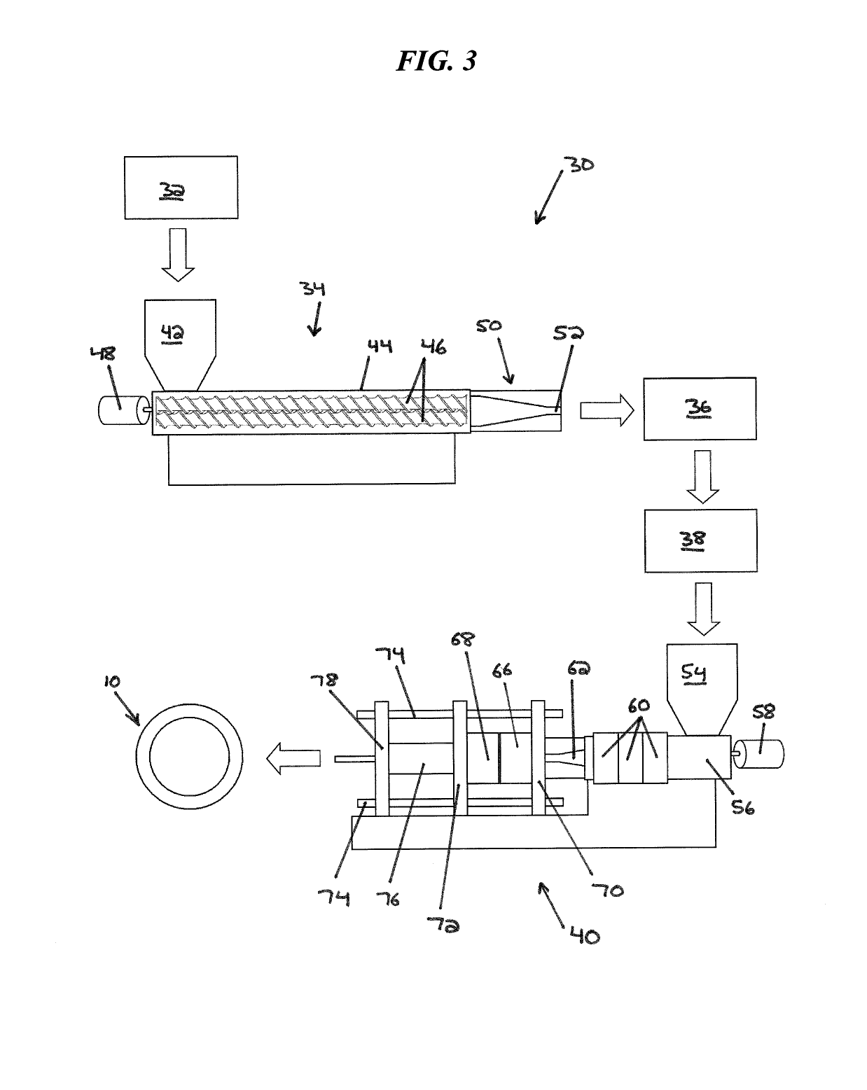

FIG. 3 is a schematic diagram of an exemplary system 30 for manufacturing a drug delivery device 10. The system 30 generally includes a mixer 32, an extrusion system 34, a pelletizer 36, a blender 38, and an injection molding system 40.

Components of the drug delivery device (e.g., polymers, active agents, excipients, and so forth) can be mixed in desired proportions using the mixer 32. Any of a variety of commercially available mixers can be used, including a GMX-Lab Micro High Shear Mixing System available from Freund-Vector Corporation of Tokyo, Japan. In an exemplary embodiment, a 750 gram batch of extrudate for forming ring-shaped vaginal drug delivery devices containing leuprolide as the active ingredient can be manufactured using the components and proportions set out below in Table 2 of the Examples section.

The resulting mixture can then be fed into the hopper 42 of the extrusion system 34. Any of a variety of commercially available extrusion systems can be used, including a Leistritz ZSE 18 HP Extruder System with an 18 mm screw diameter, and an extruder barrel L/D ratio of 25:1, available from Leistritz Corporation of Allendale, N.J. Material placed in the hopper 42 can be fed into the barrel 44 of the extrusion system 34. The barrel 44 can include one or more screws or other elements 46 rotatably mounted therein, which can be driven by a motor system 48. The barrel 44 and the elements 46 disposed therein can be selected and configured in an almost infinite number of arrangements and combinations to perform various operations, such as intake, melting, atmospheric venting, degassing, mixing, vacuum venting, metering, kneading, conveying, and so forth. Material conveyed through the barrel 44 exits the extrusion system 34 by being forced through a nozzle 50 having an ejection aperture 52 as a finished extrudate.

As noted above, a number of parameters of the extrusion system 34 can be varied to produce drug delivery devices with favorable properties. For example, the feed rate of the hopper 42 can be set so as not to overfeed the material which could unnecessarily exert additional pressure thereon and input additional energy thereto. In some embodiments, the feed rate of the hopper can be between about 0.5 kg/hr and about 2.0 kg/hr, e.g., about 0.75 kg/hr, about 1.0 kg/hr, or about 1.5 kg/hr. In some embodiments, the feed rate of the hopper can be less than about 2.0 kg/hr.

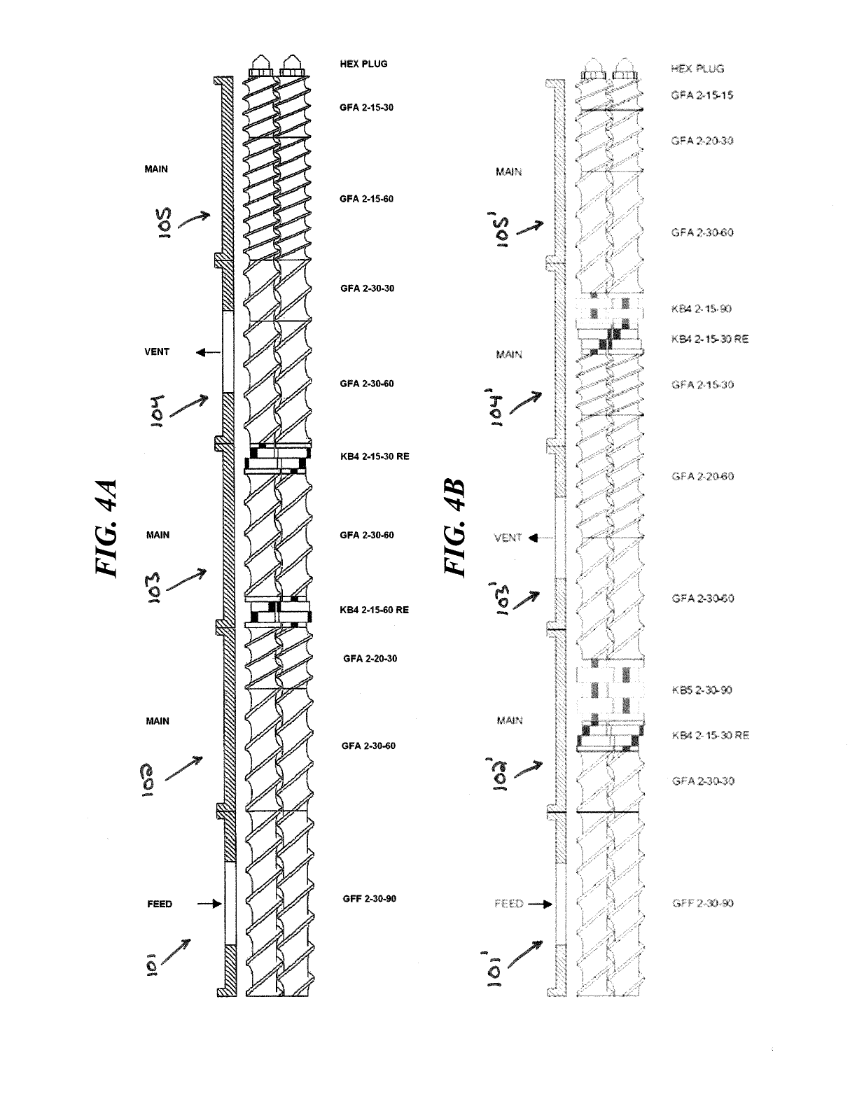

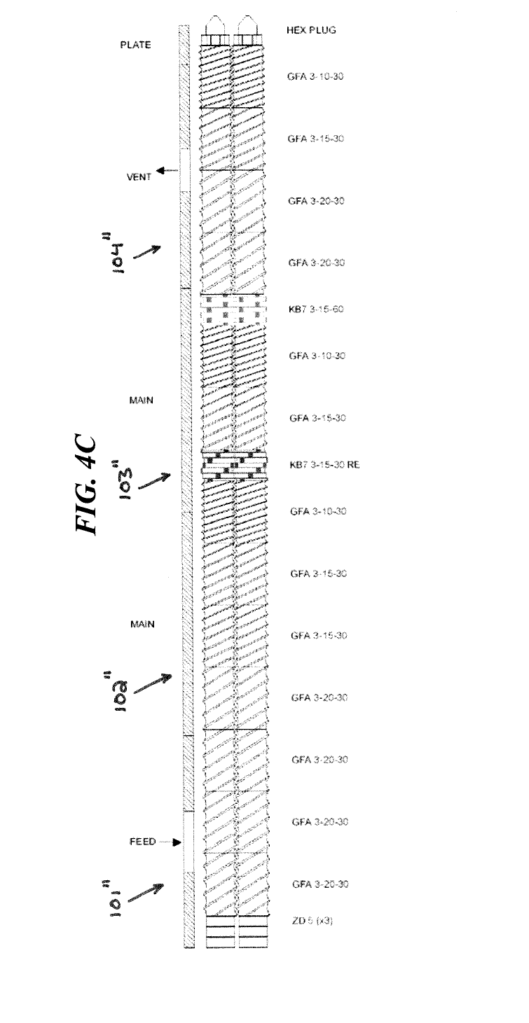

By way of further example, the barrel 44 and element 46 configuration can be selected to improve various properties of the drug delivery device. Generally speaking, the barrel and element configuration can be chosen so as to minimize the length of travel, the amount of mixing, and the amount of heat and pressure applied to the material while still providing the minimum energy required to form a clinically-useful extrudate. FIGS. 4A through 4C illustrate exemplary extruder barrel and element configurations.

The various elements in FIGS. 4A through 4C are labeled using a code system. “GFF” refers to a (G) co-rotating, (F) conveying, (F) free-cutting element. “GFA” refers to a (G) co-rotating, (F) conveying, (A) free-meshing element. “KB” refers to a kneading block type mixing element and “KB4” refers to a kneading block with 4 kneading segments. “ZD” refers to a spacer element. For the conveying elements, the numerical portion of the code refers to the number of threads, the pitch, and the length of the screw element. Thus, “GFF 2-30-90” refers to a co-rotating, conveying, and free-cutting element having 2 threads, a pitch of 30, and a length of 90 mm. For the kneading elements, the numerical portion of the code refers to the number of threads, the length of the kneading block, and the twisting angle of the individual kneading segments. The “RE” refers to a reverse-conveying element. Thus, “KB4 2-15-60 RE” refers to a reverse-conveying kneading block element with 4 kneading segments, 2 threads, a length of 15 mm, and a twisting angle of 60 degrees. For the spacer elements, the numerical portion of the code refers to the length of the spacer in millimeters.

As shown in FIG. 4A, the barrel can be arranged with a first section 101 where material feeding takes place. The first section 101 can be open, can have a length of approximately 90 mm, and can include a first element GFF 2-30-90. A second section 102 can be disposed adjacent to the first section 101. The second section 102 can be closed, can have a length of approximately 90 mm, and can include a second element GFA 2-30-60 and a third element GFA 2-20-30. A third section 103 can be disposed adjacent to the second section 102. The third section 103 can be closed, can have a length of approximately 90 mm, and can include a fourth element KB4 2-15-60 RE, a fifth element GFA 2-30-60, and a sixth element KB4 2-15-30 RE. A fourth section 104 can be disposed adjacent to the third section 103 and can allow venting to take place. The fourth section 104 can be open, can have a length of approximately 90 mm, and can include a seventh element GFA 2-30-60 and an eighth element GFA 2-30-30. A fifth section 105 can be disposed adjacent to the fourth section 104. The fifth section 105 can be closed, can have a length of approximately 90 mm, and can include a ninth element GFA 2-15-60 and a tenth element GFA 2-15-30. A final melt plate (not shown) can be disposed adjacent to the fifth section 105 and can have the extrusion nozzle 50 disposed therein or thereon.

As shown in FIG. 4B, the barrel can be arranged with a first open section 101′ where material feeding takes place, a second closed section 102′, a third open section 103′ where venting takes place, a fourth closed section 104′, and a fifth closed section 105′. The screws can be arranged with a first element GFF 2-30-90, a second element GFA 2-30-30, a third element KB4 2-15-30 RE, a fourth element KB5 2-30-90, a fifth element GFA 2-30-60, a sixth element GFA 2-20-60, a seventh element GFA 2-15-30, an eighth element KB4 2-15-30 RE, a ninth element KB4 2-15-90, a tenth element GFA 2-30-60, an eleventh element GFA 2-20-30, and a twelfth element GFA 2-15-15. A final melt plate (not shown) can be disposed adjacent to the fifth section 105 and can have the extrusion nozzle 50 disposed therein or thereon.

As shown in FIG. 4C, the barrel can be arranged with a first open section 101″ where material feeding takes place, a second closed section 102″, a third closed section 103″, and a fourth closed section 104″ where venting takes place. The screws can be arranged with a first element ZD 5 (three 5 mm spacer elements), a second element GFA 3-20-30, a third element GFA 3-20-30, a fourth element GFA 3-20-30, a fifth element GFA 3-20-30, a sixth element GFA 3-15-30, a seventh element GFA 3-15-30, an eighth element GFA 3-10-30, a ninth element KB7 3-15-30 RE, a tenth element GFA 3-15-30, an eleventh element GFA 3-10-30, a twelfth element KB7 3-15-60, a thirteenth element GFA 3-20-30, a fourteenth element GFA 3-20-30, a fifteenth element GFA 3-15-30, and a sixteenth element GFA 3-10-30. A final melt plate (not shown) can be disposed adjacent to the fifth section 105 and can have the extrusion nozzle 50 disposed therein or thereon.

Other manufacturing variables that can impact the performance of the drug delivery device include the extrusion screw speed and the extrusion temperature. The amount of force and energy applied to the material as it passes through the barrel 44 can be proportional to the speed at which the screws 46 rotate, such that a higher screw rotation speed results in a greater amount of energy and force being applied to the material. Accordingly, the screw speed can be set to a relatively low value to reduce the energy applied to the extrudate and thereby reduce the homogeneity of the extrudate. In some embodiments, the screw speed can be between about 100 rpm and about 200 rpm, e.g., about 125 rpm, about 150 rpm, or about 175 rpm. In some embodiments, the screw speed can be less than about 200 rpm. The amount of force and energy applied to the material as it passes through the barrel 44 can also be proportional to the temperature within the barrel. Accordingly, the temperature can be set to a relatively low value to reduce the energy applied to the extrudate and thereby reduce the homogeneity of the extrudate. In some embodiments, the barrel temperature can be between about 70 degrees C. and about 90 degrees C., e.g., about 75 degrees C., about 80 degrees C., or about 85 degrees C. In some embodiments, the barrel temperature can be less than about 90 degrees C.

The size chosen for the ejection aperture 52 of the nozzle 50 (e.g., the diameter or cross-sectional area of the aperture) can also affect the characteristics of the extrudate. In particular, the amount of force and energy applied to the extrudate as it passes through a relatively small aperture can be significantly higher than if it were to pass through a relatively large aperture, since the larger aperture provides less resistance and produces less turbulence. The aperture size can thus be made relatively large so as to reduce the energy applied to the extrudate and thereby reduce the homogeneity of the extrudate. In some embodiments, the nozzle aperture 52 has a circular cross section and a diameter between about 2.0 mm and about 4.0 mm, e.g., about 2.5 mm, about 3.0 mm, or about 3.5 mm. In some embodiments, the nozzle aperture 52 has a circular cross section and a diameter of at least about 3.0 mm. While the exemplary embodiments discussed above refer to an aperture with a circular cross-section, the aperture can have any of a variety of other cross-sectional shapes, e.g., elliptical, square, rectangular, and so forth. It will be appreciated that in embodiments in which a non-circular cross-section is used, the aperture can have a cross-sectional area that is about equal to that of a circle having the above-listed diameters.

In an exemplary embodiment, the barrel and element configuration shown in FIG. 4A can be used with a feed rate of about 1 kg/hr, a screw speed of about 150 rpm, a barrel temperature of about 80 degrees C., and a circular nozzle having a diameter of about 3.0 mm. Relative terms and terms of degree used herein (e.g., “about”) will be understood by those having ordinary skill in the art as referring to a range of values for which no appreciable difference in the finished drug delivery device is observed (e.g., no clinically-significant difference or no statistically-significant difference).

Referring again to FIG. 3, the extrudate can exit the extrusion system 34 onto a conveyor as a substantially continuous strand. The extrudate strand can then be fed to the pelletizer 36 to be cut into discrete units. Any of a variety of commercially-available mechanical cutting or chopping units can be used for the pelletizer, such as a Scheer Bay BT-25 Pelletizer available from Bay Plastics Machinery Company of Bay City, Mich. In some embodiments, the pelletizer can be set to separate the extruded strand into discrete units having no dimension greater than about 10 mm (e.g., no dimension greater than about 7 mm, about 5 mm, or about 3 mm).

The pelletized extrudate can then be fed to the blender 38 to be prepared for injection molding. Any of a variety of commercially-available blenders can be used for the blender, such as an 8 quart V-Shell blender available from Vanguard Pharmaceutical Machinery, Inc. of Spring, Tex. The pelletized extrudate can be mixed in the blender 38 for between 0 and 10 minutes, e.g., at least about 4 minutes, at least about 5 minutes, or at least about 6 minutes.

After being pelletized and blended, the extrudate can be supplied to the injection molding system 40 and molded into a finished drug delivery device 10. It will be appreciated that the injection molding system 40 can be a separate system at a separate location, or can be coupled to and fed directly by the other components shown in FIG. 3. The injection molding system 40 can include a hopper 54 which feeds material into a barrel 56. Conveying elements in the barrel 56 can be driven by a hydraulic pump, motor, and gear system 58 to advance the material through a series of heaters 60. The heated material is then forced through a molding nozzle 62 and into a mold defined by first and second mold plates 66, 68. The first mold plate 66 can be coupled to a stationary platen 70 and the second mold plate 68 can be coupled to a movable platen 72 which can be selectively moved along one or more tie bars 74 towards or away from the stationary platen 70. A clamping unit 76 mounted on a rear platen 78 can hold the mold plates 66, 68 together as material is forced into the mold under increased pressure and temperature. Any of a variety of commercially-available injection molding systems can be used, such as a Sesame Nano-Molder Injection Molding Machine available from LMG Corporation of De Pere, Wis.

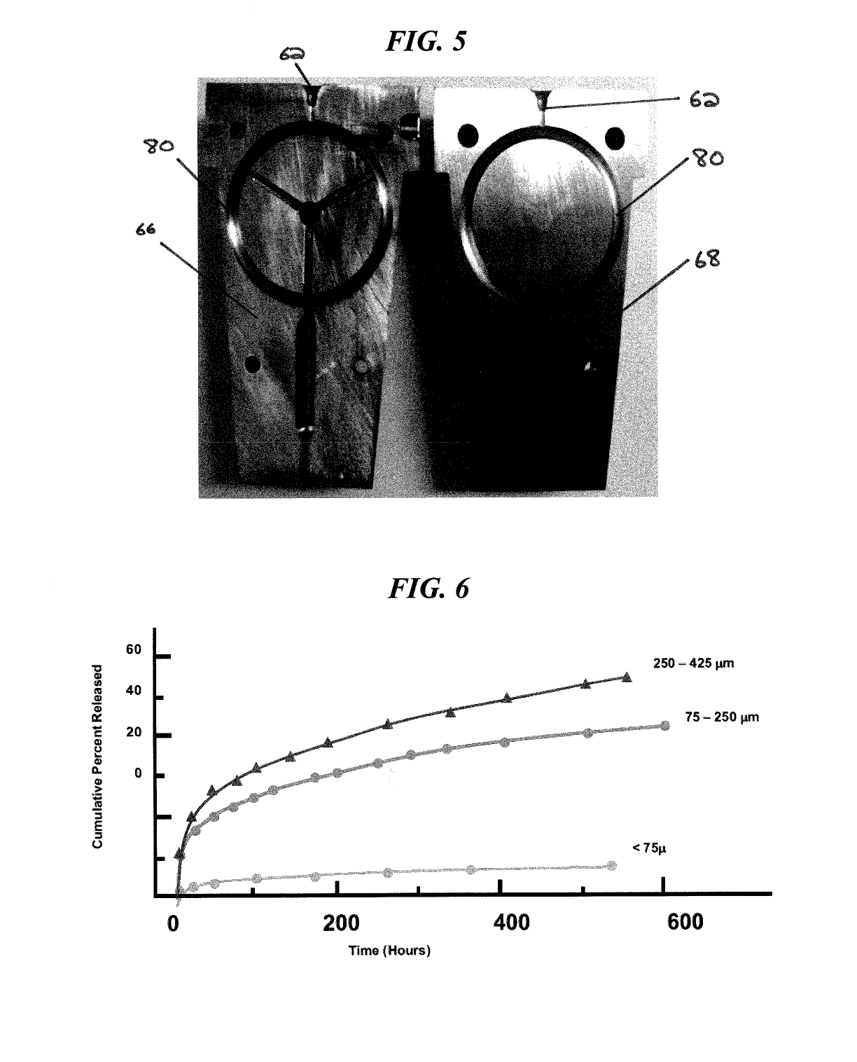

FIG. 5 illustrates an exemplary embodiment of first and second mold plates 66, 68 that can be used in the injection molding system 40 to form ring-shaped drug delivery devices (e.g., rings having a 4 mm minor diameter and a 54 mm major diameter suitable for placement in a human vagina). As shown, the first and second mold plates 66, 68 define a mold cavity 80 in which the delivery device is formed, and the nozzle 62 through which material is supplied to the mold 80. It will be appreciated that more than one mold cavity can be formed in the plates such that multiple drug delivery devices can be molded simultaneously. The additional mold cavities can share the same inlet nozzle or can each have their own independent inlet nozzle.

As in the extrusion process described above, the various operating parameters of the injection molding system 40 can be optimized to produce drug delivery devices with favorable properties. Generally speaking, reducing the energy inputs to the system (e.g., mixing, shear, cavitation, temperature, and pressure) can result in delivery devices with less homogeneity and better release characteristics.

For example, the rotational speed of the injection molding drive screw(s) can be kept low to reduce the energy and force exerted on the material. In some embodiments, the injection molding system can be configured to advance material into the mold at a rate between about 50 mm per second and about 150 mm per second, e.g., about 75 mm per second, about 100 mm per second, or about 125 mm per second. In some embodiments, the material speed can be less than about 125 mm per second.

By way of further example, the pressure setting of the injection molding system 40 can be kept low to reduce the energy and force exerted on the material. In some embodiments, the injection molding system can be configured to pressurize the material to between about 1400 bar and about 1700 bar, e.g., about 1500 bar, about 1550 bar, or about 1600 bar. In some embodiments, the injection molding pressure can be less than about 1600 bar.

By way of further example, the temperature applied to the material can be kept low to reduce the energy and force exerted on the material. In some embodiments, the temperature of the barrel 56 can be between about 75 degrees C. and about 95 degrees C., e.g., about 80 degrees C., about 85 degrees C., or about 90 degrees C. In some embodiments, the barrel temperature can be less than about 90 degrees C. In some embodiments, the temperature of the mold 80 can be between about 45 degrees C. and about 65 degrees C., e.g., about 50 degrees C., about 55 degrees C., or about 60 degrees C. In some embodiments, the mold temperature can be less than about 60 degrees C.

The size of the nozzle 62 can also be increased to reduce the amount of energy and force exerted on the material. In some embodiments, the nozzle can have a circular cross section and a diameter between about 1.5 mm and about 2.5 mm, e.g., about 1.75 mm, about 2.0 mm, or about 2.25 mm. In some embodiments, the nozzle can have a circular cross section and a diameter of at least about 1.75 mm. While the exemplary embodiments discussed above refer to an aperture with a circular cross-section, the aperture can have any of a variety of other cross-sectional shapes, e.g., elliptical, square, rectangular, and so forth. It will be appreciated that in embodiments in which a non-circular cross-section is used, the aperture can have a cross-sectional area that is about equal to that of a circle having the above-listed diameters. The nozzle diameter can also be specified relative to the diameter of the drug delivery device being manufactured. For example, the nozzle diameter can be selected to be approximately one third, one half, or two thirds the size of the product diameter.

In an exemplary embodiment, the injection molding system 40 can be configured with a circular nozzle having a diameter of about 2.0 mm, a molding pressure of about 1550 bar, a speed setting of about 100 mm per second, a mold temperature of about 55 degrees C., and a barrel temperature of about 85 degrees C. Relative terms and terms of degree used herein (e.g., “about”) will be understood by those having ordinary skill in the art as referring to a range of values for which no appreciable difference in the finished drug delivery device is observed (e.g., no clinically-significant difference or no statistically-significant difference).

Once the injection molding process is complete, the finished drug delivery device 10 can be cooled, removed from the mold, and packaged for use.

As evident from the foregoing, the processing conditions used to manufacture drug delivery devices can have as much if not more of an impact on release characteristics as the specific formulation used for the drug delivery device. Surface area can also affect release characteristics but can be nearly insignificant relative to the impact of processing conditions. Energy inputs (mixing, shear, cavitation, temperature, pressure, and so forth) play a critical role in device performance and ultimate success.

FIGS. 6-10 illustrate how the release kinetics of the drug delivery device can be affected by the composition and formulation of the device.

FIG. 6 illustrates the relationship between the release kinetics/cumulative release of the delivery device and the particle size of the drug loaded therein. Larger drug particle sizes increase the contact between particles within the device and thus increase the creation of tortuous pathways through which other particles can pass. Thus, as shown, larger drug particle sizes result in faster release kinetics. In particular, drug particle sizes between 250 and 425 μm are released faster than drug particle sizes between 75 and 250 μm, which are released faster than drug particle sizes that are less than 75 μm. As also shown, higher cumulative release is achieved when larger drug particles are used.

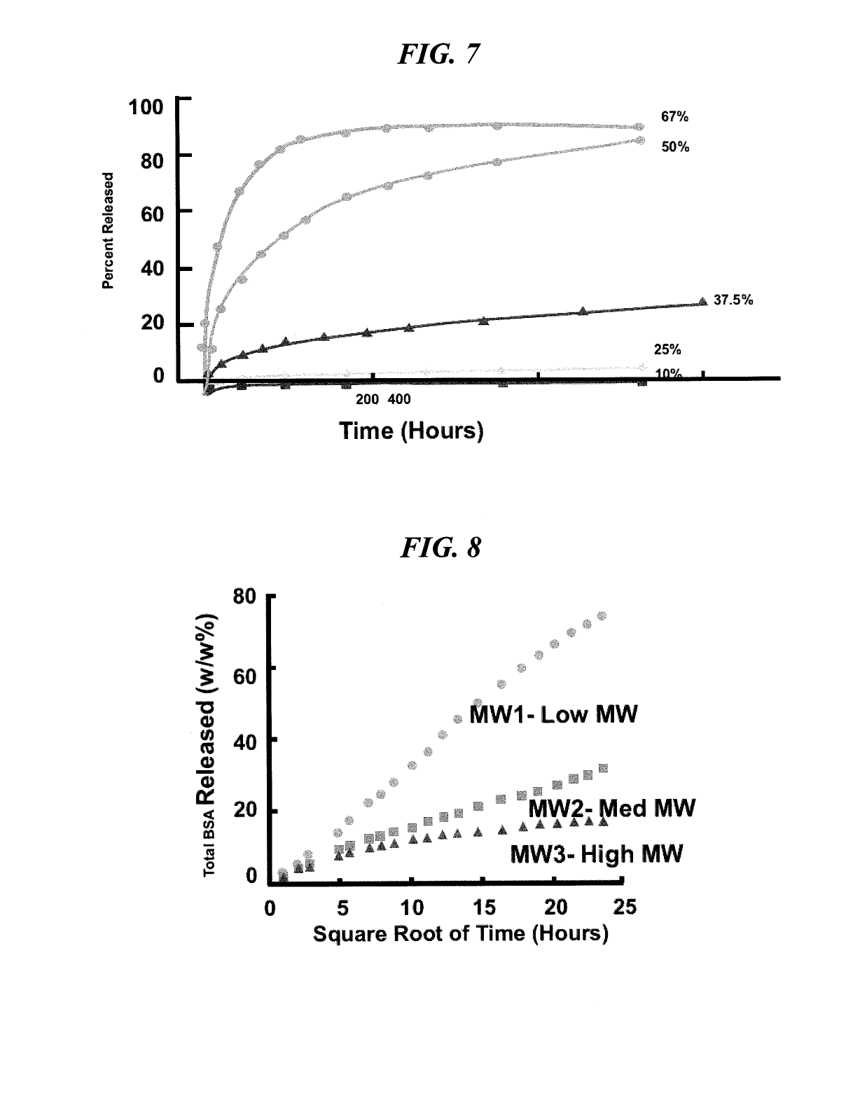

FIG. 7 illustrates the relationship between release kinetics/cumulative release of the delivery device and the loading of the drug contained therein. Higher loading increases the contact between particles within the device and thus increases the creation of tortuous pathways through which other particles can pass. Thus, as shown, higher drug loading results in faster release kinetics. In particular, devices with a 67% drug loading release the drug faster than devices with a 50% drug loading, which release the drug faster than devices with a 37.5% drug loading, which release the drug faster than devices with a 25% drug loading, which release the drug faster than devices with a 10% drug loading. As also shown, higher cumulative release is achieved with increased loading.

FIG. 8 illustrates the relationship between release kinetics of the delivery device and the molecular weight of the drug loaded therein. Higher molecular weight drugs have more difficulty traversing through the polymer and are more susceptible to entrapment which slows the release rate. Thus, as shown, higher molecular weight results in slower release kinetics. In particular, low molecular weight drugs are released faster than medium molecular weight drugs, which are released faster than high molecular weight drugs.

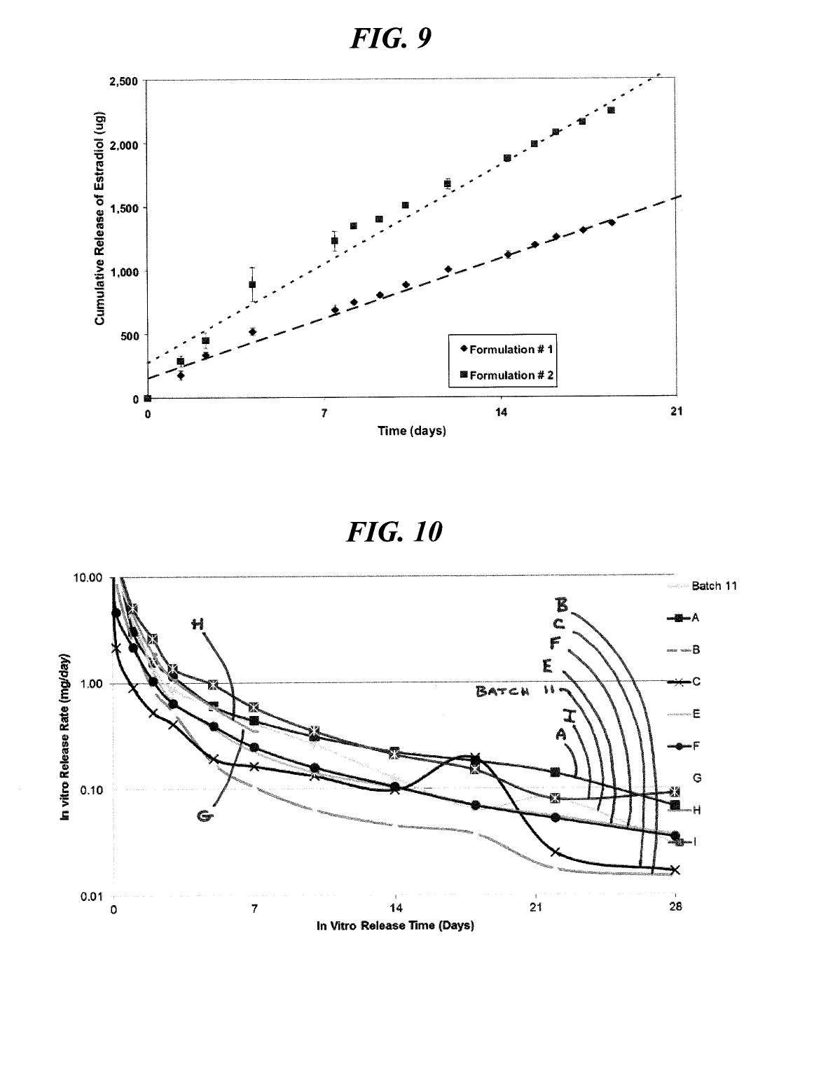

FIG. 9 illustrates the relationship between release kinetics of the delivery device and the excipients included in the device formulation. A first formulation (“Formulation #1”) using a first excipient produced a device with faster release kinetics than a second formulation (“Formulation 2”) which used a second, different excipient. As shown, Estradiol is soluble in EVA and can be released with pseudo zero order release kinetics.

FIG. 10 illustrates the relationship between release kinetics of the delivery device and the formulation used when the drug is a peptide such as leuprolide acetate. The batch 11 formulation was a control that included extrudate by PharmaForm, and rings injected molded by Bionex. Formulations A, B, C, E, F, G, H, and I are set out by weight percentage in Table 1 below. As shown, Formulation I included citric acid, which provided good mechanical properties and higher release rate throughout. Formulation A included higher amounts of PEG4000 and a blend of EVA polymers that yielded similar properties (release and mechanical) with improved shelf life.

TABLE 1

| | Lot No. | | | | | | | |
|---|---|---|---|---|---|---|---|---|
| | CB101201A | CB101201B | CB101201C | CB101215A | CB101215B | CB110105A | CB110105B | NF3 |
| | Formulation ID | | | | | | | |
| | A | B | C | E | F | G | H | I |
| Ingredient Name | % | % | % | % | % | % | % | % |
| Leuprolide Acetate | 2.4 | 2.4 | 2.4 | 2.4 | 2.4 | 2.4 | 2.4 | 2.4 |
| PEG 4000 | 8 | 4 | | 4 | | | | 4 |
| PEG 8000 | | | 4 | | 4 | 4 | 4 | |
| Tween 80 | 1 | | | 1 | 1 | 1.5 | 2.0 | 1 |
| Citric Acid | | 10 | 10 | 10 | 10 | 10 | 10 | 10 |
| Evatane 18-150 | 44.3 | 41.8 | 41.8 | 41.3 | 41.3 | 49.26 | 48.96 | 82.6 |
| Evatane 28-25 | 44.3 | 41.8 | 41.8 | 41.3 | 41.3 | 32.84 | 32.64 | |
| Total | 100 | 100 | 100 | 100 | 100 | 100.00 | 100.00 | 100 |

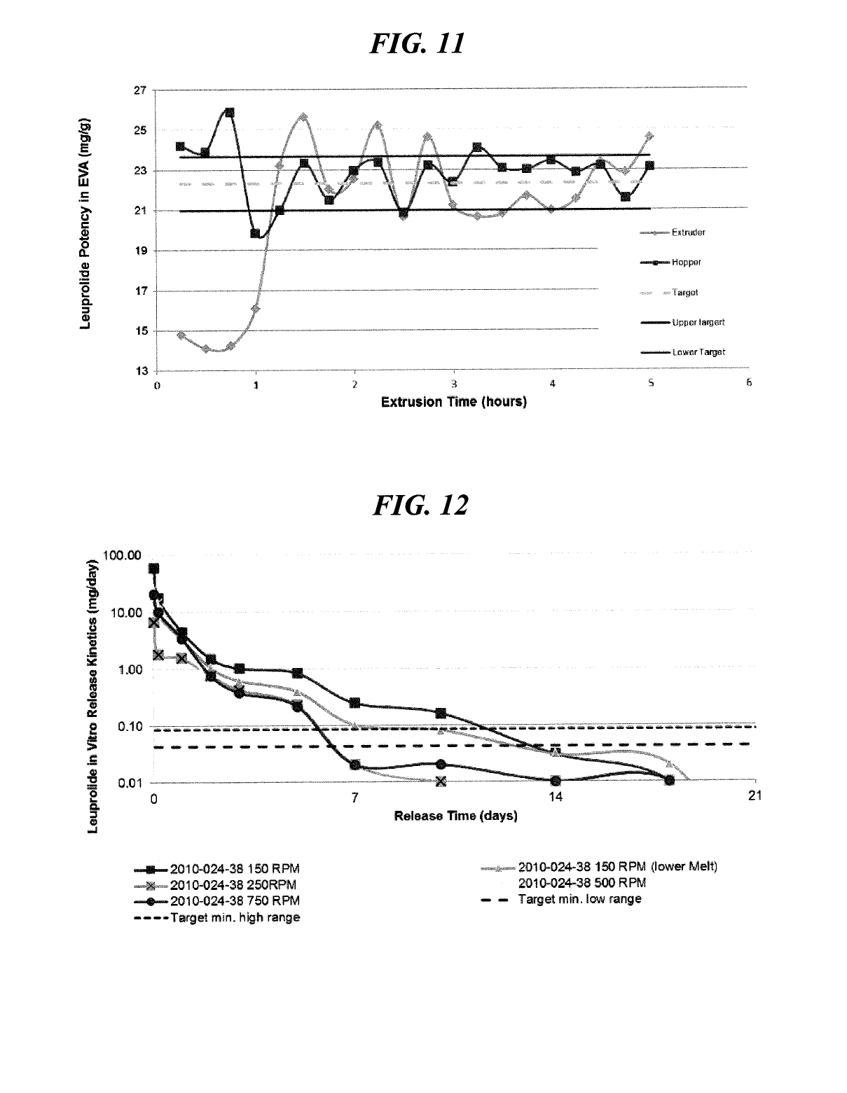

FIGS. 11-15 illustrate how various properties of the device can be affected by the manufacturing parameters that are employed.

FIG. 11 illustrates the drug potency as a function of extrusion time for a delivery device that uses EVA for the polymer and leuprolide for the drug. Initially, the leuprolide tends to coat the interior of the hopper and the various other parts of the extrusion system, resulting in a decreased potency within the polymer. After about an hour, however, the coatable parts of the extrusion system become saturated and the potency within the polymer increases. As extrusion continues, the leuprolide potency in the extruder and hopper stabilizes within the target range.

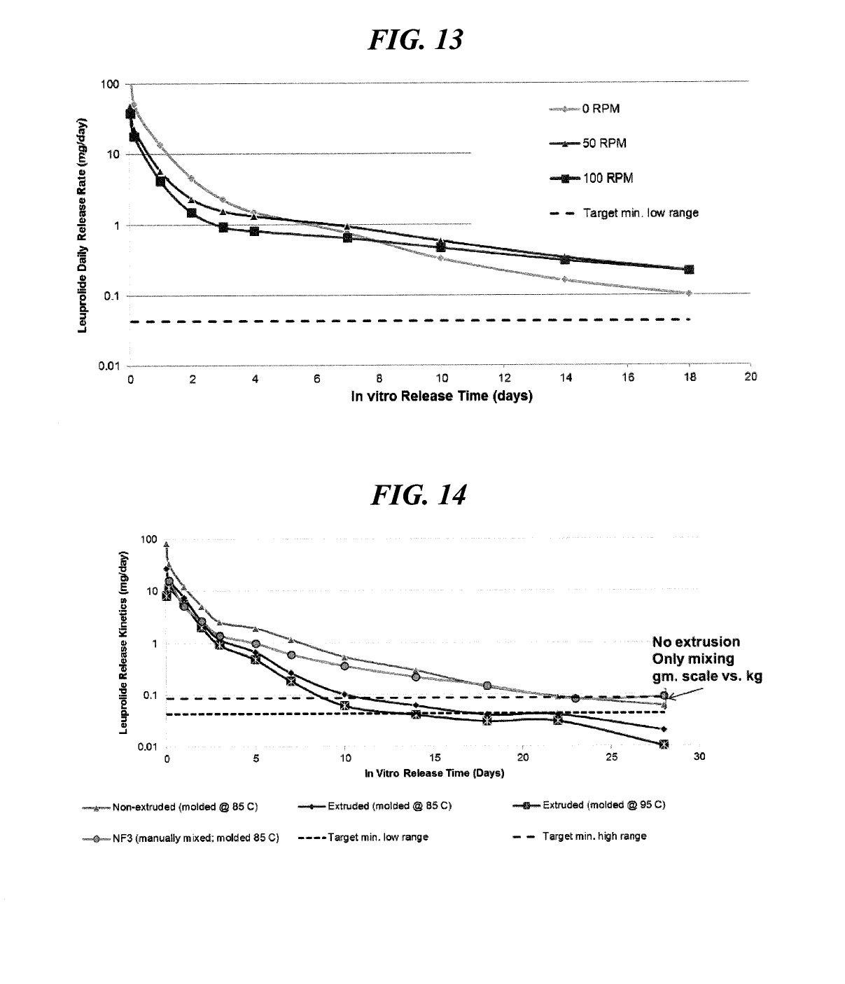

FIGS. 12 and 13 illustrate the relationship between the release kinetics of the delivery device and the rotational speed of the extrusion screws when the device was manufactured. Higher RPM screw speeds increase the energy and force exerted on the material, and thus increase the homogeneity of the device which can inhibit the creation of tortuous release pathways. Thus, as shown in FIG. 12, lower RPM screw speeds can result in faster release kinetics as well as a longer overall release period than the same formulation manufactured using higher RPM screw speeds. In particular, a screw speed of 150 rpm produced faster release kinetics and a longer release period than the same formulation when screw speeds of 250, 500, and 750 rpm were used. As shown in FIG. 13, the lower RPM settings (e.g., 0 rpm, 50 rpm, 100 rpm) resulted in delivery devices with favorable release profiles. The standard deviation for the release kinetics at 0 rpm was 50%, whereas the standard deviation at 50 rpm and 100 rpm was less than 10%.

FIG. 14 illustrates the relationship between the release kinetics of the delivery device and the molding temperature used when the device was manufactured. Lower molding temperatures reduce the energy and force exerted on the material, and thus decrease the homogeneity of the device which can encourage the creation of tortuous release pathways. Thus, lower molding temperatures can result in faster release kinetics as well as a longer overall release period than the same formulation manufactured using higher molding temperatures. In particular, non-extruded devices molded at a temperature of 85 degrees C. produced faster release kinetics and a longer release period than the same formulation extruded and molded at 85 degrees C. and the same formulation extruded and molded at 95 degrees C.

FIG. 15 illustrates the relationship between finished device stiffness (compression force as a function of compression distance when the ring is deformed into an oval shape) and the manufacturing process used to produce the device. As shown, extruded devices generally have a higher stiffness than non-extruded devices. Using the manufacturing methods disclosed herein, drug delivery devices can be produced which are stiff enough to be retained in the vaginal cavity for extended periods of time yet compliant enough to be comfortable for the patient. The 11C17 extruded device, produced using the methods disclosed herein, has favorable strength properties but is soft enough to not abrade tissue in the vaginal cavity. In addition to improved stiffness, the methods disclosed herein can also produce rings with stronger knit-lines (the location where material injected into a ring shaped mold at a single inlet meets, e.g., 180 degrees opposite from the inlet location). The methods disclosed herein can also reduce the amount of air bubbles visible in the finished drug delivery device, as well as the amount of polymer “hairs” visible on the exterior of the outer edge (“parting line blow through”).

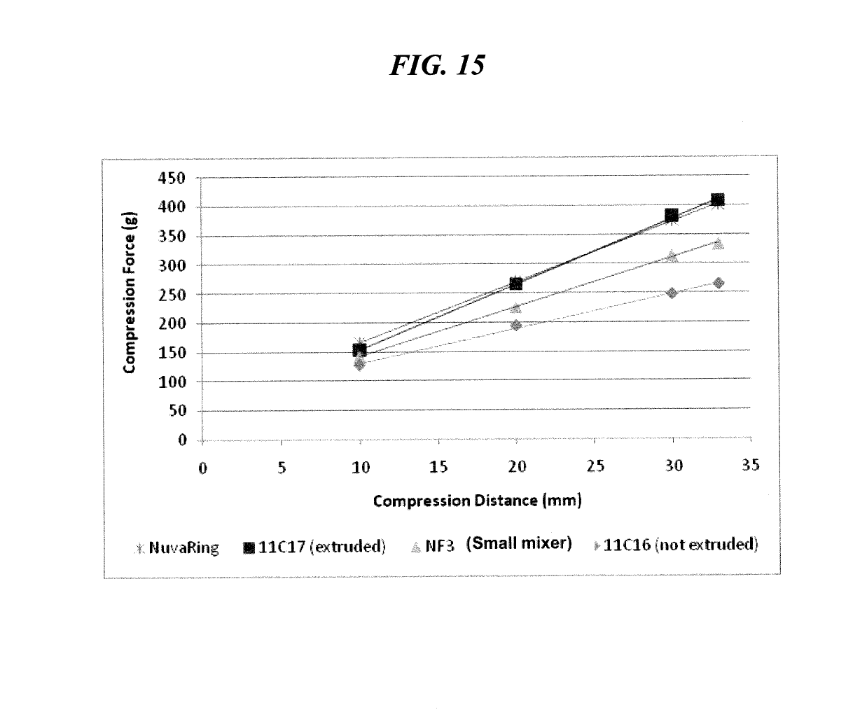

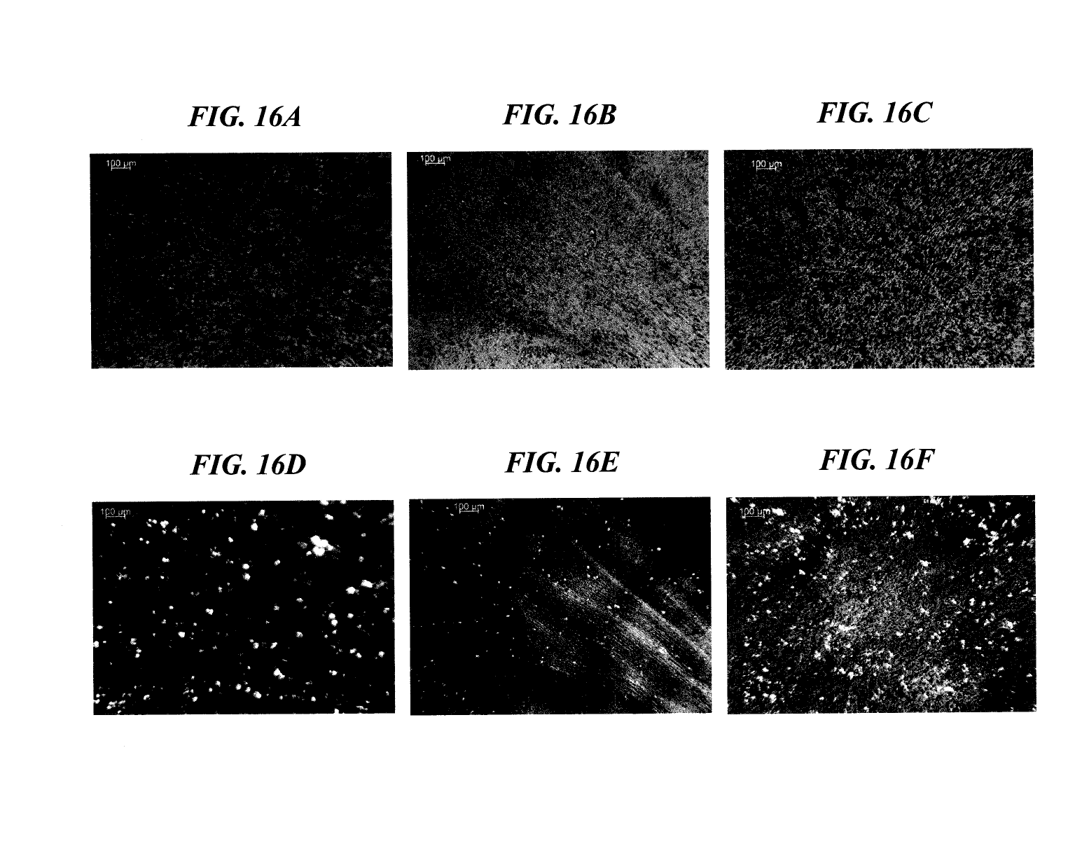

FIGS. 16A-16F are optical microscope images of drug delivery devices produced using various manufacturing techniques. FIG. 16A is a common transmission image of raw extrudate (i.e., before molding) manufactured as described herein. FIG. 16D is a polarized light image of the same raw extrudate. FIG. 16B is a common transmission image of a drug delivery device manufactured using high pressure injection molding. FIG. 16E is a polarized light image of the same device. FIG. 16C is a common transmission image of a drug delivery device manufacturing using low pressure injection molding. FIG. 16F is a polarized light image of the same device. As shown, injection molding, which involves additional energy being applied to the material, increases the homogeneity of the composition and the isolation of the drug particles as compared with non-molded materials (e.g., the raw extrudate). Furthermore, higher molding pressures, which again involve additional energy being applied to the material, increase the homogeneity of the composition and the isolation of the drug particles as compared with devices manufactured using lower molding pressures. FIGS. 16A-16F thus demonstrate that manufacturing with lower energy inputs can result in drug delivery devices with decreased homogeneity and increased particle contact, which can lead to more favorable release properties.

When two or more agents are to be delivered, the foregoing steps of manufacture can be repeated for each individual agent, thus forming, for example, a separate molded polymeric mixture for each agent. The individual molded polymeric mixtures can be cut into pieces of the required length using conventional cutting techniques, thus producing a plurality of uniform segments. The drug delivery device for simultaneous delivery of multiple agents, or for delivery, e.g., of an antiandrogen and one or more contraceptive agents can be then assembled by joining together, directly or indirectly, at least one segment of the molded polymeric mixture for each agent to be delivered. The uniform segments can be assembled to form a ring shape, which can have a thickness between about 1 mm and about 5 mm. It will be appreciated that drug delivery devices can be manufactured in a wide range of shapes, sizes, and forms for delivering the active agent(s) to different environments of use.

Alternatively, when one, two, or more active agents are to be delivered, each active agent/polymer mix can be molded together to the desired shape, through injection, compression, and/or extrusion such that the one or two agent mixtures form one solid unit and do not require a coupling means. In some embodiments, the agent mixtures can be injected, preferably sequentially, into a mold comprising a single inlet port. In other embodiments, the active agent mixtures can be injected simultaneously or sequentially into a mold having multiple inlet ports. Multiple port moldings are well known and commercially-available in the art. Such molding can be modified or customized for a particular application as will be appreciated by those of skill in the art.

In some embodiments, the ends of the segments can be joined together to form a drug delivery device using a coupling means. The coupling means can be any method, mechanism, device, or material known in the art for bonding materials or structures together. Exemplary coupling means include solvent bonding, adhesive joining, heat fusing, heat bonding, pressure, and the like. When a solvent is used, the ends of the segments can be moistened with an organic solvent that causes the surfaces to feel tacky, and when placed in contact the surfaces then bond and adhere in a fluid tight union. The ends of the segments can be adhesively united to form a ring-shaped delivery device by applying an adhesive to at least one end of a segment, and then contacting the adhesive coated end or ends. For the above procedures, the solvents include organic solvents such as methylene chloride, ethylene dichloride, trichlorobenzene, dioxan, isophorone, tetrahydrofuran, aromatic and chlorinated hydrocarbons, mixed solvents such as 50/50 ethylene dichloride/diacetone alcohol; 40/60 alcohol/toluene; 30/70 alcohol/carbon tetrachloride, and the like. Suitable adhesives include natural adhesives and synthetic adhesives, such as animal, nitrocellulosic, polyamide, phenolic, amino, epoxy, isocyanate, acrylic, silicate, organic adhesives of polymers, and the like. Adhesives are well known to the art (see, e.g., The Encyclopedia of Chemistry (Second ed.; G. L. Clark and G. G. Hawley, editors; VanNostrand Reinhold Co., Cincinnati, Ohio; 1966), as well as solvents (see, e.g., Encyclopedia of Chemical Technology (Kirk-Othmer, Sec. Ed., Vol. 16, Interscience, Publishers Inc., New York, 1969)).

The lengths of the segments of the drug delivery device can be chosen to give the required performance. Ratios of the lengths of the segments will depend upon the particular therapeutic application, including the desired ratio and dosages of each active agent to be delivered. Ratios of the lengths of the segments can be between 30:1 and 1:30, for example between about 15:1 and 1:1. Placebo segments can be used to prevent active agent diffusion and interactions, e.g., when two or more active agents are used, and the lengths of the placebo segments can be long enough to prevent excessive mixing of the active agents. The length of the placebo segments can depend on the nature of the polymeric substance and its capacity to prevent permeation of the active agents. In some embodiments, the placebo segment can completely or substantially prevent mixing of the active agents, since mixing can disturb the release pattern. However, depending upon which active agent is used, some minor mixing can generally be permitted, provided it does affect the release of the active agents in such a manner that plasma levels of the active agents do not substantially exceed the required values.

Intravaginal drug delivery devices disclosed herein can be manufactured in any size as required. The cross sectional diameter of polymer rods can typically be between about 0.5 mm and 12 mm, between 0.5 mm and 10 mm, between 1 mm and 8 mm, or even between 1 and 6 mm, for example between 1 and 5 mm. In the case of human use, the ring-shaped device can have an outer diameter from about 40 mm to about 80 mm; the cross sectional diameter can preferably be between about 0.5 mm to 12 mm.

In an exemplary embodiment, a drug delivery device can be a vaginal ring having about 15 to about 18 g of EVA (e.g., about 17 g) (for example, about 8.5 grams of EVA having about 28 weight percent vinyl acetate, and a melt index at 190 degrees C./2.16 kg of 28 g/10 nm, and about 8.5 grams of EVA having about 18 weight percent weight percent vinyl acetate, and a melt index at 190 degrees C./2.16 kg of about 150 g/10 nm), and about 1.67% grams of PEG having a wt. average molecular weight of about 4000, about 0.2 grams of Tween 80, and about 0.7 grams of leuprolide acetate.

In some embodiments, the device can have substantially no vinyl acetate monomers, e.g., less than about 1, 0.5, or even less than about 0.05 weight percent vinyl acetate monomer.

Methods of Treatment

A variety of treatment methods are possible with the drug delivery devices disclosed herein. For example, methods for vaginally delivering therapeutic agents to a female mammal are provided in which a drug delivery device as described above is positioned in the vaginal tract of the female mammal to be treated, where it is maintained for a period of time sufficient to deliver a pharmaceutically-effective amount of one or more active agents to the female mammal. The pharmaceutically-effective amount of one or more active agents can be less than the pharmaceutically-effective amount when said one or more active agents is administered to a patient orally. Such methods can result in reduced incidence of adverse side-effects in patients as compared to oral administration. For example, such methods can result in reduced incidence of gastrointestinal side effects in patients as compared to oral administration.

The devices disclosed herein can also allow direct administration of the one or more active agents to a target organ without initial metabolism by the liver. The methods and devices disclosed herein can be used to treat and/or ameliorate obesity, diabetes, multiple sclerosis, endometriosis, polycystic ovarian disease, uterine fibroids, breast cancer, hirsutism, acne, microbial infections (e.g., bacterial vaginosis), coronary heart disease, chronic obstructive pulmonary disease, asthma, chronic kidney disease, and/or migraine. In some embodiments, the drug administration methods provide a continuous, simultaneous delivery of physiological combinations of therapeutic agents without the need for injections and/or vaginal gels or creams.

A dose range of a therapeutic agent can depend upon the particular composition used. As will be understood by one of skill in the art, the effective dose ranges can be agent specific and can depend upon patient characteristics, such as species, age, and weight. An effective dose range can be determined by routine testing by one of skill in the art, without undue experimentation. For example, an effective dose of one or more contraceptive agents can together provide substantial protection from pregnancy. In another example, an effective dose of one or more cholesterol lowering agents can together provide substantial reduction of blood cholesterol levels.

Additional details on treating a patient using with a vaginal drug delivery device are disclosed in U.S. Patent Application No. 2011/0280922, entitled “DEVICES AND METHODS FOR TREATING AND/OR PREVENTING DISEASES,” which is hereby incorporated by reference in its entirety.

Prescribing, Marketing, or Sales Methods

Despite the numerous advantages of using a vaginal drug delivery device as opposed to alternative administration methods and devices, they have not been universally adopted. This may be due, in part, to the difficulty in determining which patients would be willing to try a vaginal drug delivery device or would prefer a vaginal drug delivery device. The present applicant has discovered that women who trim or shave their pubic area are far more likely to use vaginal drug delivery devices than women who do not. It is believed that approximately 25% of women over the age of 18 trim their pubic hair and that approximately 32% of women over the age of 18 shave their pubic hair. It is further believed that women aged 18-44 are more likely to shave than older women.

In some embodiments, a method of prescribing, marketing, or selling a vaginal drug delivery device is provided. The method can include determining whether a patient can benefit from vaginal administration of one or more drugs, determining whether a pubic area of the patient has been shaved or trimmed, and prescribing, marketing, or selling a vaginal drug delivery device to the patient if the pubic area of the patient has been shaved or trimmed. In some embodiments, a method of prescribing, marketing, or selling a vaginal drug delivery device is provided that includes prescribing, marketing, or selling the vaginal drug delivery device to a patient if a pubic area of the patient has been shaved or trimmed. In some embodiments, a method of treatment is provided that includes administering a vaginal drug delivery device (e.g., as described herein) to a patient that can benefit from vaginal administration of one or more drugs, if a pubic area of the patient has been shaved or trimmed.

### EXAMPLES

### Example 1

A 750 gram batch of Leuprolide Acetate Extruded Pellets, 2.4% w/w was manufactured using the components out in Table 2 below:

TABLE 2

| | | | Theoretical | Amount to |
|---|---|---|---|---|
| | | % w/w of | amount for | Dispense |
| | Component | Extrudate | 750 g | g |
| 1 | Ethylene Vinyl Acetate | 44.3 | 332.25 | 332.25 |
| | Copolymer, Milled | | | |
| | (Evatane EVA 28-40, Milled) | | | |
| 2 | Polyethylene Glycol, NF | 8.0 | 60.00 | 60.00 |
| | (PEG 4000, Granular, NF) | | | |
| 3 | Polysorbate 80, NF | 1.0 | 7.50 | 7.575 |
| 4 | Ethylene Vinyl Acetate | 44.3 | 332.25 | 332.25 |
| | Copolymer, Milled | | | |
| | (Evatane EVA 18-150, Milled) | | | |
| 5 | Leuprolide Acetate | 2.4 | 18.00 | 18.00 |
| | Total Extrusion Blend | 100.0 | 750 | 750.075 |

The listed components were dispensed according to the mass listed in the far right column. 7.575 grams of Polysorbate 80, NF was dispensed into a 60 mL syringe. A 1% excess of the Polysorbate 80, NF over the theoretical amount of 7.5 grams was dispensed to allow for residual losses in the syringe.

Primary Mixing:

30 grams of the Polyethylene Glycol, NF was placed in the bowl of a GMX-Lab Micro High Shear Mixing System with a 1 liter bowl and 1 liter blades. The GMX-Lab Micro was closed with the chopper set to OFF. The Polyethylene Glycol, NF was then mixed with a plow speed of 155 RPM for approximately 30 seconds. 18 grams of the Leuprolide Acetate was then added into the 1 liter bowl of the GMX-Lab Micro. The remaining 30 grams of the Polyethylene Glycol, NF was used to rinse the liner from the Leuprolide Acetate into the 1 liter bowl of the GMX-Lab Micro. The GMX-Lab Micro was then closed with the chopper set to OFF and the Polysorbate 80, NF was slowly charged from the syringe into the GMX-Lab Micro bowl while mixing with a plow speed of 155 RPM. The combination was mixed for approximately 3 minutes.

Secondary Mixing:

332.25 grams of Ethylene Vinyl Acetate Copolymer, Milled (Evatane EVA 18-150, Milled) was then added into the bowl of a GMX-Lab Micro High Shear Mixing System with a 4 liter bowl and 4 liter blades (1st Layer). The Polyethylene Glycol, NF, Leuprolide Acetate, and Polysorbate 80, NF mixture was then collected from the GMX-Lab Micro 1 liter bowl and transferred to the 4 liter bowl of the GMX-Lab Micro (2^{nd} Layer). 332.25 grams of Ethylene Vinyl Acetate Copolymer, Milled (Evatane EVA 28-40, Milled) was then added into the 4 liter bowl of the GMX-Lab Micro (3rd Layer). The GMX-Lab Micro was closed and the combination was mixed for approximately 5 minutes at 425 rpm with the chopper set to ON, low speed range. The blend was then transferred into a container double lined with poly bags with 2 desiccant, 4 unit silica gel between the inner and outer liner.

Hot Melt Extrusion Procedure:

A Leistritz ZSE 18 HP Extruder System with a 25:1 extruder barrel was arranged with the following barrel configuration: Open Barrel (Feed); Closed Barrel; Closed Barrel; Open Barrel (Vent); Closed Barrel; Final Melt Plate. The 25:1 length/diameter ratio twin screws were assembled as shown in FIG. 4A and installed into the extruder. A 3.0 mm single bore round die and spacer was installed onto the final melt plate. Supply and return connections were made between a Tempered Water Generator (TWG) and the extruder. In particular, tempered water lines were connected to the extrusion barrel manifold, one set of cooling water lines was connected to the feeding barrel, one set of cooling lines was connected to the gear box, and all supply and return valves were placed in the open position.

The TWG and pump were turned on, the chilled water set point was adjusted to 13.0° C., and the tempered water set point was adjusted to 30.0° C. A K-Tron KCL24T20 Feeder w/12 mm diameter 20 pitch screws was connected to the extruder and positioned behind the extruder. The K-Tron was bonded/grounded to the extruder and the impeller inside of the feeder hopper was installed so as not to touch the wall of the hopper. The extruder was turned on and the temperature set points for each heating zone were set according to Table 3 below:

TABLE 3

| | Feeding | | | | | |
|---|---|---|---|---|---|---|
| Zone | Zone | 1 | 2 | 3 | 4 | (Melt Plate) |
| Set Point (° C.) | N/A | 80 | 80 | 80 | 80 | Record |
| | | | | | | Actual |
| Set Point | N/A | ±10 | ±10 | ±10 | ±10 | N/A |
| Range (° C.) | | | | | | |
| ON/OFF | OFF | ON | ON | ON | ON | OFF |

A 30 minute wait time was observed to allow the extruder to reach thermal equilibrium. The K-Tron feeding method was set to normal and the K-Tron feeder hopper was filled with the blend from the GMX-Lab Micro described above. The feed rate was set to 0.75 kg/hr and the K-Tron was run until the material began to flow to prime the feed screws. The K-Tron's auto calibration routine was then executed and the K-Tron was aligned with the extruder feed opening. The K-Tron feeder was set to Gravimetric Dosing mode and the feed rate set point for the extruder was set at 100% for feeder 1, and 0.0% for feeder 2. A Dorner Cooling Conveyer was aligned with the extruder die with cooling fan 1 off and fans 2, 3, and 4 on.

The feed opening of a Scheer Bay BT-25 Pelletizer was aligned at the end of the cooling conveyor. A container double lined with poly bags with 2 desiccant, 4 unit silica gel between the inner and outer liner was placed beneath the pelletizer discharge chute to collect the pelletized material.

The screw drive was started at 10.0 rpm and the screws were allowed to turn a few revolutions to ensure proper installation. The screw speed was then increased to 50 rpm (±10 rpm) and feeder 1 was started at 100% of the feed rate total for an effective feed rate of 0.75 kg/hr (±0.2) kg/hr. The cooling conveyer was bypassed and the extruder was run into a waste container for 5 minutes, or until the extrudate was translucent with no dark spots. The conveyer and pelletizer speeds were adjusted to draw the extrudate in such a way as to produce pellets approximately 5 mm or smaller. The product funnel on the extruder was monitored to ensure buildup of material did not occur. If excessive build-up was observed, excess material was vacuumed from the product funnel area. During the batch, the heated zone set points were adjusted within the ranges noted in Table 3 above to maintain the target temperature.

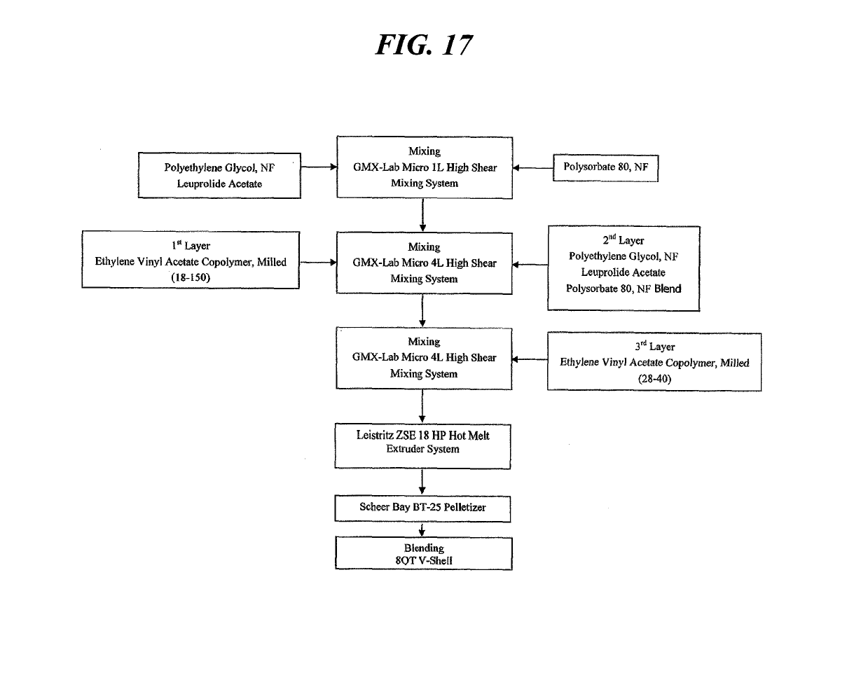

The extrudate was visually examined to verify that the extrudate was translucent with no dark spots. If extrudate appearance became opaque or output decreased, the melt plate was heated to 80° C. to 90° C. using a torch. The K-Tron feeder hopper was refilled as necessary and set to the Gravimetric Dosing mode after each re-fill. The pelletized extrudate was added into an 8 quart V-Shell blender and blended for 5 minutes (±1 minute). The blended extrudate was then transferred into a container double lined with poly bags with 2 desiccant, 4 unit silica gel between the inner and outer liners. The procedure to this point is illustrated schematically in FIG. 17.

Injection Molding Procedure

The pelletized and blended extrudate was then transferred to the hopper of a Sesame Nano-Molder Injection Molding Machine configured with the mold plates of FIG. 5 which defined a mold cavity with a 4 mm minor ring diameter and a 54 mm major ring diameter. A ring-shaped drug delivery device was injection molded using a 2.0 mm circular nozzle, a molding pressure of 1550 bar, a speed setting of 100 mm per second, a mold temperature of 55 degrees C., and a barrel temperature of 85 degrees C. The finished drug delivery device was then allowed to cool and removed from the mold.

### Example 2

Leuprolide acetate was dispensed under appropriate containment in the quantity specified in Table 2 above and blended with PEG 4000 and polysorbate 80 in a GMX-Lab Micro High Shear Mixing System. The mixture was then transferred to a GMX laboratory scale granulator. Milled EVA 28-40 and milled EVA 18-150 were dispensed in the quantity specified in Table 2 above and transferred to the GMX laboratory scale granulator and mixed with the leuprolide acetate blend for 5 minutes at settings of 950 rpm and 3600 rpm for the impeller and chopper.

The mixture was then added to the extrusion system, which was configured as described above in Example 1. Material was gravimetrically fed into the extruder at a feed rate of 1 kg/hr while the extruder was heated to 80 degrees C. and while the extruder screws rotated at 150 rpm. All material discharged from the extruder die was collected on the cooling conveyor and directly pelletized. Pelletized material was collected in double polyethylene bags. The pelletized material (Leuprolide Acetate Extruded Pellets, 2.4% w/w Bulk Drug) was then fed into the barrel of a Sesame Nano-Molder Injection Molding Machine and injection molded according to the procedure used in Example 1 above. The formed Leuprolide EVA rings were placed in a tray or on a clean wipe.

### CONCLUSION

Although the invention has been described by reference to specific embodiments, it should be understood that numerous changes may be made within the spirit and scope of the inventive concepts described. For example, while a number of vaginal ring embodiments are described above, the drug delivery devices disclosed herein can be formed in any of a variety of shapes for placement in any of a variety of locations within or on a human or animal body. Accordingly, it is intended that the invention not be limited to the described embodiments, but that it have the full scope defined by the language of the following claims.

What is claimed is:

1. A method of manufacturing a drug delivery device, comprising:
mixing one or more drugs, one or more excipients, and one or more polymers to form a mixture;
extruding the mixture using an extrusion system to form an extrudate; and
injection molding at least a portion of the extrudate into a drug delivery device having a predetermined shape using an injection molding system;
wherein the mixture is at least one of:
extruded through the extrusion system using an extrusion screw rotation speed between about 100 rpm and about 200 rpm,
extruded through the extrusion system using a barrel temperature between about 70 degrees C. and about 90 degrees C.,
molded at a pressure between about 1400 bar and about 1700 bar, and
injected into a mold through a nozzle having a cross-sectional area equal to that of a circle having a diameter between about 1.5 mm and about 2.5 mm.

2. The method of claim 1, wherein the one or more drugs comprises leuprolide acetate, the one or more excipients comprises Polysorbate 80, and the one or more polymers comprises ethylene-vinyl-acetate (EVA) copolymer and polyethylene glycol (PEG).

3. The method of claim 2, wherein the one or more polymers comprises EVA 28-40, EVA 18-150, and PEG 4000.

4. The method of claim 1, wherein the mixture comprises EVA 28-40 at a weight percentage of 44.3, PEG 4000 at a weight percentage of 8.0, Polysorbate 80 at a weight percentage of 1.0, EVA 18-150 at a weight percentage of 44.3, and leuprolide acetate at a weight percentage of 2.4.

5. The method of claim 1, wherein the mixture is fed into the extrusion system at a rate of about 0.5 kg/hr to about 2.0 kg/hr.

6. The method of claim 1, wherein the mixture is fed into the extrusion system at a rate of less than about 2.0 kg/hr.

7. The method of claim 1, wherein the mixture is fed into the extrusion system at a rate of about 1.0 kg/hr.

8. The method of claim 1, wherein the mixture is extruded through the extrusion system using a barrel configuration that includes a first open section where the mixture is fed, a second closed section, a third closed section, a fourth open section where venting occurs, and a fifth closed section.

9. The method of claim 1, wherein the mixture is extruded through the extrusion system using an element configuration of GFF 2-30-90 at the feed, followed by GFA 2-30-60, followed by GFA 2-20-30, followed by KB4 2-15-60 RE, followed by GFA 2-30-60, followed by KB4 2-15-30 RE, followed by GFA 2-30-60, followed by GFA 2-30-30, followed by GFA 2-15-60, followed by GFA 2-15-30.

10. The method of claim 1, wherein the mixture is extruded through the extrusion system using an extrusion screw rotation speed between about 100 rpm and about 200 rpm.

11. The method of claim 1, wherein the mixture is extruded through the extrusion system using an extrusion screw rotation speed less than about 200 rpm.

12. The method of claim 1, wherein the mixture is extruded through the extrusion system using an extrusion screw rotation speed of about 150 rpm.

13. The method of claim 1, wherein the mixture is extruded through the extrusion system using a barrel temperature between about 70 degrees C. and about 90 degrees C.

14. The method of claim 1, wherein the mixture is extruded through the extrusion system using a barrel temperature less than about 90 degrees C.

15. The method of claim 1, wherein the mixture is extruded through the extrusion system using a barrel temperature of about 80 degrees C.

16. The method of claim 1, wherein the mixture is extruded through a nozzle having a cross-sectional area equal to that of a circle having a diameter between about 2.0 mm and about 4.0 mm.

17. The method of claim 1, wherein the mixture is extruded through a nozzle having a cross-sectional area equal to that of a circle having a diameter of at least about 2.5 mm.

18. The method of claim 1, wherein the mixture is extruded through a nozzle having a cross-sectional area equal to that of a circle having a diameter of about 3.0 mm.

19. The method of claim 1, wherein the mixture is fed into the extrusion system at a rate of about 1.0 kg/hr, is extruded through the extrusion system using an extrusion screw rotation speed of about 150 rpm, is extruded through the extrusion system using a barrel temperature of about 80 degrees C., and is extruded through a nozzle having a cross-sectional area equal to that of a circle having a diameter of about 3.0 mm.

20. The method of claim 1, further comprising pelletizing the extrudate before said injection molding.

21. The method of claim 20, further comprising blending the extrudate after said pelletizing and before said injection molding.

22. The method of claim 1, wherein the extrudate is advanced into a mold of the injection molding system at a rate between about 50 mm per second and about 150 mm per second.

23. The method of claim 1, wherein the extrudate is advanced into a mold of the injection molding system at a rate less than about 125 mm per second.

24. The method of claim 1, wherein the extrudate is advanced into a mold of the injection molding system at a rate of about 100 mm per second.

25. The method of claim 1, wherein the extrudate is molded at a pressure between about 1400 bar and about 1700 bar.

26. The method of claim 1, wherein the extrudate is molded at a pressure less than about 1600 bar.

27. The method of claim 1, wherein the extrudate is molded at a pressure of about 1550 bar.

28. The method of claim 1, wherein the extrudate is molded using a barrel temperature between about 75 degrees C. and about 95 degrees C.

29. The method of claim 1, wherein the extrudate is molded using a barrel temperature less than about 90 degrees C.

30. The method of claim 1, wherein the extrudate is molded using a barrel temperature of about 80 degrees C.

31. The method of claim 1, wherein the extrudate is molded using a mold temperature between about 45 degrees C. and about 65 degrees C.

32. The method of claim 1, wherein the extrudate is molded using a mold temperature less than about 60 degrees C.

33. The method of claim 1, wherein the extrudate is molded using a mold temperature of about 55 degrees C.

34. The method of claim 1, wherein the extrudate is injected into a mold through a nozzle having a cross-sectional area equal to that of a circle having a diameter between about 1.5 mm and about 2.5 mm.

35. The method of claim 1, wherein the extrudate is injected into a mold through a nozzle having a cross-sectional area equal to that of a circle having a diameter of at least about 1.75 mm.

36. The method of claim 1, wherein the extrudate is injected into a mold through a nozzle having a cross-sectional area equal to that of a circle having a diameter of about 2.0 mm.

37. The method of claim 1, wherein the extrudate is advanced into a mold of the injection molding system at a rate of about 100 mm per second, the extrudate is molded at a pressure of about 1550 bar, the extrudate is molded using a barrel temperature of about 80 degrees C., the extrudate is molded using a mold temperature of about 55 degrees C., and the extrudate is injected into a mold through a nozzle having a cross-sectional area equal to that of a circle having a diameter of about 2.0 mm.

38. The method of claim 1, wherein the predetermined shape comprises a ring.

39. The method of claim 1, wherein the ring has a minor diameter of about 4 mm and a major diameter of about 54 mm.

40. A drug delivery device, comprising:
a ring-shaped body comprising leuprolide acetate, Polysorbate 80, ethylene-vinyl-acetate (EVA) copolymer, and polyethylene glycol (PEG);
wherein, when placed in a vaginal tract of a patient, the ring-shaped body is configured to release the leuprolide acetate at a rate of at least about 0.1 mg/day for a period of at least about 10 days.

41. The drug delivery device of claim 40, wherein, when placed in the vaginal tract of the patient, the ring-shaped body is configured to release the leuprolide acetate at a rate of at least about 0.15 mg/day.

42. The drug delivery device of claim 40, wherein, when placed in the vaginal tract of the patient, the ring-shaped body is configured to release the leuprolide acetate for a period of at least about 28 days.

43. A drug delivery device, comprising:
a ring-shaped body comprising a mixture of one or more drugs, one or more excipients, and one or more polymers;
wherein the body is formed using an extrusion process followed by an injection molding process, the extrusion process or the injection molding process including at least one of:
extruding the mixture using an extrusion screw rotation speed between about 100 rpm and about 200 rpm,
extruding the mixture using a barrel temperature between about 70 degrees C. and about 90 degrees C.,
molding the mixture at a pressure between about 1400 bar and about 1700 bar, and
injecting the mixture into a mold through a nozzle having a cross-sectional area equal to that of a circle having a diameter between about 1.5 mm and about 2.5 mm.

44. The device of claim 43, wherein the one or more drugs comprises leuprolide acetate, the one or more excipients comprises Polysorbate 80, and the one or more polymers comprises ethylene-vinyl-acetate (EVA) copolymer and polyethylene glycol (PEG).

45. The device of claim 43, wherein the one or more polymers comprises EVA 28-40, EVA 18-150, and PEG 4000.

46. The device of claim 43, wherein the body comprises EVA 28-40 at a weight percentage of 44.3, PEG 4000 at a weight percentage of 8.0, Polysorbate 80 at a weight percentage of 1.0, EVA 18-150 at a weight percentage of 44.3, and leuprolide acetate at a weight percentage of 2.4.

47. The device of claim 43, wherein the extrusion process includes feeding the mixture into an extrusion system at a rate of about 0.5 kg/hr to about 2.0 kg/hr.

48. The device of claim 43, wherein the extrusion process includes extruding the mixture using a barrel configuration that includes a first open section where the mixture is fed, a second closed section, a third closed section, a fourth open section where venting occurs, and a fifth closed section.

49. The device of claim 43, wherein the extrusion process includes extruding the mixture using an element configuration of GFF 2-30-90 at the feed, followed by GFA 2-30-60, followed by GFA 2-20-30, followed by KB4 2-15-60 RE, followed by GFA 2-30-60, followed by KB4 2-15-30 RE, followed by GFA 2-30-60, followed by GFA 2-30-30, followed by GFA 2-15-60, followed by GFA 2-15-30.

50. The device of claim 43, wherein the extrusion process includes extruding the mixture using an extrusion screw rotation speed between about 100 rpm and about 200 rpm.

51. The device of claim 43, wherein the extrusion process includes extruding the mixture using a barrel temperature between about 70 degrees C. and about 90 degrees C.

52. The device of claim 43, wherein the extrusion process includes extruding the mixture through a nozzle having a cross-sectional area equal to that of a circle having a diameter between about 2.0 mm and about 4.0 mm.

53. The device of claim 43, wherein the injection molding process includes advancing the mixture into a mold at a rate between about 50 mm per second and about 150 mm per second.

54. The device of claim 43, wherein the injection molding process includes molding the mixture at a pressure between about 1400 bar and about 1700 bar.

55. The device of claim 43, wherein the injection molding process includes molding the mixture using a barrel temperature between about 75 degrees C. and about 95 degrees C.

56. The device of claim 43, wherein the injection molding process includes molding the mixture using a mold temperature between about 45 degrees C. and about 65 degrees C.

57. The device of claim 43, wherein the injection molding process includes injecting the mixture into a mold through a nozzle having a cross-sectional area equal to that of a circle having a diameter between about 1.5 mm and about 2.5 mm.

58. The device of claim 43, wherein the body has a minor diameter of about 4 mm and a major diameter of about 54 mm.
